SEQUENCE LISTING (18 sequences)

SEQ ID NO: 1 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 2 - protein, 3144 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 3 - protein, 140 aa, Homo sapiens
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQY

SEQ ID NO: 4 - protein, 526 aa, Homo sapiens
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPY

SEQ ID NO: 5 - protein, 386 aa, Homo sapiens
MEDEMPKTLYVGNLSRDVTEALILQLFSQIGPCKNCKMIMDTAGNDPYCFVEFHEHRHAAAALAAMNGRKIMGKEVKVNWATTPSSQKKDTSSSTVVSTQRSQDHFHVFVGDLSPEITTEDIKAAFAPFGRISDARVVKDMATGKSKGYGFVSFFNKWDAENAIQQMGGQWLGGRQIRTNWATRKPPAPKSTYESNTKQLSYDEVVNQSSPSNCTVYCGGVTSGLTEQLMRQTFSPFGQIMEIRVFPDKGYSFVRFNSHESAAHAIVSVNGTTIEGHVVKCYWGKETLDMINPVQQQNQIGYPQPYGQWGQWYGNAQQIGQYMPNGWQVPAYGMYGQAWNQQGFNQTQSSAPWMGPNYGVQPPQGQNGSMLPNQPSGYRVAGYETQ

SEQ ID NO: 6 - protein, 776 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSATKQVQRRPPPAGPRSERGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 294 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 8 - protein, 393 aa, Homo sapiens [sequence omitted]

SEQ ID NO: 9 - protein, 928 aa, Homo sapiens
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQRTPRKSNLDEEVNVIPPHTPVRTVMNTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTSQNLDSGTDLSFPWILNVLNLKAFDFYKVIESFIKAEGNLTREMIKHLERCEHRIMESLAWLSDSPLFDLIKQSKDREGPTDHLESACPLNLPLQNNHTAADMYLSPVRSPKKKGSTTRVNSTANAETQATSAFQTQKPLKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNKEEK

SEQ ID NO: 10 - protein, 586 aa, Homo sapiens
MATATPVPPRMGSRAGGPTTPLSPTRLSRLQEKEELRELNDRLAVYIDKVRSLETENSALQLQVTEREEVRGRELTGLKALYETELADARRALDDTARERAKLQIELGKCKAEHDQLLLNYAKKESDLNGAQIKLREYEAALNSKDAALATALGDKKSLEGDLEDLKDQIAQLEASLAAAKKQLADETLLKVDLENRCQSLTEDLEFRKSMYEEEINETRRKHETRLVEVDSGRQIEYEYKLAQALHEMREQHDAQVRLYKEELEQTYHAKLENARLSSEMNTSTVNSAREELMESRMRIESLSSQLSNLQKESRACLERIQELEDLLAKEKDNSRRMLTDKEREMAEIRDQMQQQLNDYEQLLDVKLALDMEISAYRKLLEGEEERLKLSPSPSSRVTVSRASSSRSVRTTRGKRKRVDVEESEASSSVSISHSASATGNVCIEEIDVDGKFIRLKNTSEQDQPMGGWEMIRKIGDTSVSYKYTSRYVLKAGQTVTIWAANAGVTASPPTDLIWKNQNSWGTGEDVKVILKNSQGEEVAQRSTVFKTTIPEEEEEEEEAAGVVVEEELFHQQGTPRASNRSCAIM

SEQ ID NO: 11 - RNA, 21 nt, Artificial Sequence
  Duplex #1
ccgaugagacugccgccaauu

SEQ ID NO: 12 - RNA, 21 nt, Artificial Sequence
  Duplex #1
uuggcggcagucucaucgguu

SEQ ID NO: 13 - RNA, 21 nt, Artificial Sequence
  Duplex #2
ggcaggacgagcauggcuauu

SEQ ID NO: 14 - RNA, 21 nt, Artificial Sequence
  Duplex #2
uagccaugcucguccugccuu

SEQ ID NO: 15 - RNA, 21 nt, Artificial Sequence
  Duplex #3
ccggaggaguggucgcaguuu

SEQ ID NO: 16 - RNA, 21 nt, Artificial Sequence
  Duplex #3
acugcgaccacuccuccgguu

SEQ ID NO: 17 - RNA, 21 nt, Artificial Sequence
  Duplex #4
caaguuuccuccucccuguuu

SEQ ID NO: 18 - RNA, 21 nt, Artificial Sequence
  Duplex #4
acagggaggaggaaacuuguu

(19) United States
(12) Patent Application Publication
(10) Pub. No.: US 2021/0121438 A1
(43) Pub. Date: Apr. 29, 2021

(54) METHODS FOR TREATING OR PREVENTING CONFORMATION DISEASES AND METHODS FOR DRUG SCREENING

(71) Applicant: GARAGE BRAIN SCIENCE CO., LTD, Taichung City (TW)

(72) Inventors: Hsiang-Yu CHANG, Taichung City (TW); I-Fan WANG, Taichung City (TW); Tsung-Yu TSAI, Arcadia, CA (US)

(21) Appl. No.: 17/256,136

(22) PCT Filed: Jun. 28, 2019
(86) PCT No.: PCT/US2019/039643

§ 371 Date: Dec. 24, 2020

Related U.S. Application Data
(60) Provisional application No. 62/691,142, filed on Jun. 28, 2018.

(51) Int. Cl. A61K 31/352; A61K 31/713; A61K 31/4545; A61K 45/06

(52) U.S. Cl. CPC A61K 31/352; A61K 45/06; A61K 31/4545; A61K 31/713

(57) ABSTRACT

Described herein are pharmaceutical compositions and methods for treating and preventing conformational diseases such as, TDP-43 proteinopathies, SMA, amyloid positive cancer, normal and premature aging. Also disclosed are in vitro screening methods for screening a therapeutic candidate to treat conformation diseases, by measuring the expression level of prion-like folding of aggregation-prone proteins or a P53 aggregate.

### CROSS-REFERENCE TO RELATED APPLICATION

This application claims the benefit of U.S. Application No. 62/691,142, filed on 28 Jun. 2018, the entire disclosure of which is incorporated herein by reference.

### TECHNOLOGY FIELD

The present invention relates to pharmaceutical compositions and methods for the treatment and prevention of conformational diseases, such as proteinopathies, neurodegenerative diseases, amyloid positive cancer, normal aging and premature aging, non-amyloidogenic and amyloidogenic diseases by stabilizing biological multivalent form, reducing protein degradation or misfolded aggregates, or increasing prion-like conformer of prion-like LC proteins.

### BACKGROUND OF THE INVENTION

Conformational disorders cause a wide variety of human diseases, particularly neurodegenerative diseases.

Age-related dementia and neurodegenerative diseases, such as limbic-predominant age-related TDP-43 encephalopathy (LATE), Alzheimer's disease, amyotrophic lateral sclerosis (ALS), frontotemporal lobar degeneration with ubiquitin-positive inclusions (FTLD-U), spinal muscular atrophy (SMA) and brain injury are chronic diseases causing major public health problems worldwide. Neurodegenerative diseases profoundly affect patients as well as their families and friends.

Misfolded protein aggregates are a leading cause of neurodegeneration, and the co-occurrence of multiple neurodegenerative proteinopathies is frequently observed in patients with dementia. Therapies to directly restore the bio-activity of misfolded disease proteins of mixed neuropathology are not yet available.

A subtype of intrinsically disordered proteins harbors low-complexity (LC) regions, similar to those causing yeast prion formation, and was recently identified by us and other groups. A computer algorithm based on protein sequence similarity to yeast prions has predicted that over 250 human proteins harbor a distinctive prion-like segment, including several RNA-binding proteins associated with neurodegenerative diseases, i.e., TDP-43 (SEQ ID No. 1), Htt (SEQ ID No. 2), PFN1 (SEQ ID No. 3), FUS (SEQ ID No. 4), and TIA1 (SEQ ID No. 5). These domains are typically rich in uncharged amino acids (Q, N, Y, S, and G), have flexible structures and can form cross-β polymers. The prion-like domain has been proposed to play a variety of roles in normal biology, such as organizing membrane-less granules, alternative splicing and heterochromatin formation, through temporal homo- and hetero-cross-β polymerization (prion-like interactions).

The structural plasticity of PLD allows for conformational conversion and transient, reversible aggregation into liquid-like phase-separated compartments, i.e., membrane-less organelles through prion-like cross-β polymerization following environmental stimulation. The ability of the prion-like protein to self-polymerize and to undergo multiple interactions with other components indicates its function as a molecular scaffold.

Ubiquitinated, phosphorylated TDP-43 C-terminus forms toxic inclusions that were originally found in the brains of patients with FTLD-U and ALS. TDP-43 pathology was later also detected in 90% of hippocampal sclerosis (HS) cases and approximately 30% of Alzheimer's disease (AD) cases using antibodies specific against abnormal phosphor-epitopes of TDP-43. Latest study suggested a common TDP-43 proteinopathy strikes after 80 years old.

The deposition of cleaved, ubiquitinated, hyperphosphorylated pathological TDP-43C-terminal fragments was found in the cytoplasm of neurons and glia in patients with ALS and FTLD.

Approximately 50 causative mutations of ALS were found in the C-terminus, which further supports a direct disease causal role for this protein.

TDP-43 is a ubiquitously expressed nuclear protein that binds to both DNA and RNA and regulates many aspects of biological processes, including polymerase II-dependent transcription, premRNA splicing, microRNA biogenesis and protein translation. Various functions of TDP-43 are involved in neurite outgrowth, axonal transport, the cell cycle and apoptosis.

The majority of TDP-43 proteins appears in the nucleus and shuttle between the nucleus and cytosol to traffic RNAs. With structural and functional resemblance to prion-like RNA-binding proteins, TDP-43 contains two RNA-binding domains and a prion-like low-complexity domain (LC Domain) at its C-terminus, which can assemble into cross-β polymers via self-intra or self-intermolecular interactions.

Self-associations of PLD are required for the formation of TDP-43 nuclear bodies, alternative splicing of CFTR and protein stability of TDP-43. Dysfunctional self-interaction leads to the degradation and misfolding of TDP-43 and is a potential etiology of TDP-43 proteinopathies.

Currently, most clinical trial research involving misfolded disease proteins aims to remove the burden of amyloid depositions via the activation of a protein degradation system, immunotherapy or inhibition of disease protein synthesis because effectively reduced toxicity of misfolded protein aggregates has been shown to slow the pathological decline in mouse models. However, in addition to removing misfolded protein aggregates, in the case of TDP-43 pathology, rescuing the physiological functions of TDP-43 is a critical determinant of therapeutic efficiency because the loss of TDP-43 cellular functions leads to abnormalities in the cell cycle and causes neurodegeneration in flies, fish, and rodents. Notably, Defective TDP-43 disrupted pre-mRNA alternative splicing and induced transposable element mis-regulation have been observed in patients with TDP-43 pathology.

Spinal muscular atrophy (SMA) is an autosomal recessive neuromuscular disorder that affects approximately 1 in 1000 babies born worldwide each year. A deficiency in the SMN protein results in a gradual loss of motor neurons in the anterior horn of the spinal cord and subsequent system-wide atrophy of skeletal muscles. The gene responsible for SMA, survival motor neuron 1 (SMN1) (SEQ ID No. 7), has been identified.

A nearly identical copy of the gene, SMN2, is normally expressed in all patients with SMA. Although a small amount of full-length protein is produced that is identical to SMN1, the exon-splicing silencer bearing a C-to-T transition in exon 7 of SMN2 in all individuals skips exon 7. An increased copy number of SMN2 modulates the severity of SMA but does not fully compensate for the loss of SMN1.

The protein product SMNA7 appears to be unstable and rapidly degrades, and its biological functions remain obscure. Although SMN1 was identified as the mutant gene responsible for SMA 20 years ago, the molecular mechanisms by which the exon 7 deletion alters cellular functions and SMA-associated mutations trigger the disease remain a mystery.

Tumor suppressor genes, including p53 (SEQ ID No. 8) and RB 1 (SEQ ID No. 9), normally act to inhibit the cell proliferation and maintain genomic integrity. Mutation in tumor suppressor genes lead to cancer. They protect a cell from one step on the path to cancer.

p53 aggregates have been experimentally shown to form amyloid oligomers and fibrils similar to those identified in Alzheimer's disease, Parkinson's disease and prion diseases, which have beta-sheet registry amyloid structures due to binding to thioflavin T.

Misfolded p53 aggregates are commonly observed in malignant tumors, particularly in chemotherapy-treated tumors or highly metastatic cancers bearing p53 mutations. Thirty to forty percent of p53-associated cancer mutations affect the structure of the protein, resulting in increased propensity toward aggregation. Currently known p53 aggregate-positive cancer types include breast, colon, skin, ovarian and prostate cancers.

p53 proteins are homotetrameric tumor suppressors that are frequently inactivated by mutation, deletion or misfolding in the majority of human tumors. p53 proteins play key roles in regulating a number of cellular processes, such as DNA repair, cell cycle control, apoptosis and senescence.

There is strong clinical correlation between misfolded p53 aggregates and cancer invasion and chemoresistance.

Despite advances made in the diagnosis and treatment of conformational disease over the last 50 years, the medical community is still faced with the challenge of treating numerous types of conformational diseases. Accordingly, there is still a need for a more effective treatment for conformational diseases. The present invention addresses this need and other needs.

### BRIEF SUMMARY OF THE INVENTION

In one embodiment, the present invention provides a pharmaceutical composition. Advantageously, the pharmaceutical combination reduces TDP-43 misfolded aggregates and/or TDP-43 and SMN degraded fragments by synergistic effects.

In another embodiment, methods for preventing or treating a conformational disease in a subject are provided, comprising administering to the subject in need thereof an effective amount of a therapeutic agent to increase the level of the prion-like folding of an aggregation-prone protein or reduce the degraded fragment or misfolded aggregate of a prion-like LC protein, wherein the symptom or sign of the conformation disease is reduced.

Also provided are in vitro methods for identifying a therapeutic candidate to treat a conformational disease, comprising the steps of a) determining the expression level of P53 aggregate in one or more test cells prior to contacting the therapeutic candidate with the one or more test cells; and b) determining the expression level of P53 aggregate in step (a) after contacting the therapeutic candidate with one or more test cells, wherein a decrease of P53 aggregate expression level after contacting the therapeutic candidate with one or more test cells relative to the P53 aggregate expression level prior to contacting the therapeutic candidate with one or more test cells, is an indication that the therapeutic candidate is efficacious for treating the conformational diseases.

Further provided are in vitro method for identifying a therapeutic candidate to treat a conformational disease, comprising the steps of a) determining the expression level of the polymer specific to the conformational disease selected from in one or more test cells prior to contacting the therapeutic candidate with the one or more test cells; and b) determining the expression level of the polymer in step (a) after contacting the therapeutic candidate with one or more test cells, wherein the polymer is selected from the group consisting essentially of TDP-43, Htt, Lamin B1, FUS, TIA-1, Tau (SEQ ID No. 6), SMN, p53, Rb (Rb1), PFN1 and SMN and an increase level of the polymer after contacting the therapeutic candidate with one or more test cells relative to the polymer expression level prior to contacting the therapeutic candidate with one or more test cells, is an indication that the therapeutic candidate. The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

The invention will become more apparent when read with the accompanying figures and detailed description which follow.

### BRIEF DESCRIPTION OF THE DRAWINGS

Illustrative embodiments of the present invention are described in detail below with reference to the following Figures:

FIG. 1 is an assembly of images illustrating baicalein remodels TDP-43 fibers into TDP-43 polymers in vitro Panel (a) and (b) contains Purified full-length TDP-43 recombinant proteins (equivalent 3 μM monomer) were incubated in the absence (a) or presence of baicalein (3 μM; b) assembled buffers at RT agitated for 30 min, 60 min, and 90 min following by validation with electron microscopy. Arrowheads in b indicated representative high-magnification images of TDP-43 polymers in lower panel. One of branching points was indicated by arrow. Bars in a: 1 μm; Bars in b: 0.5 Panel (c) contains statistical analysis of the length of TDP-43 fibers and TDP-43 polymers. All data are presented as the mean with SD (n=6). Panel (d) contains a bar graph of illustrating branching point analysis of baicalein-inducing TDP-43 polymers. All data are presented as the mean with SD (n=10). Panel (e) is two selected electron micrographs of negatively stained structures of polymerizing TDP-43. Scale bars: 100 nm.

FIG. 2 is an assembly of images illustrating off-amyloid pathway compounds reduced pathological-like TDP-43 inclusion and increased solubility. Panel (a) contains 293T cells with TDP-43-IIPLD were treated with 50 μM of baicalein or no baicalein. Two images of individual treatments are shown. Arrowhead indicated TDP-43-IIPLD aggregates. Bars: 10 μm. Panel (b) contains the statistical analysis of effectiveness of baicalein on TDP-43-IIPLD aggregates is shown. All data are presented as the mean with SD (n=3). *P<0.05 by t-test. Panel (c) is a photograph of a western blot showing the effectiveness of baicalein in enhancing the solubility of TDP-43-IIPLD post-treatment with low-dose or high-dose baicalein for 48 or 9 h, respectively. Panel (d) illustrates 293T cells with TDP-43-IIPLD were treated with 50 μM EGCG. Two images of individual treatments are shown. Bars: 10 Panel (e) contains images of EGCG disassembled the formed TDP-43 misfolded aggregates in a dose-dependent manner. All data are presented as the mean with SD (n=3). *P<0.05 by t-test. Panel (f) is a photograph of a western blot showing TDP-43-IIPLD in urea fractions post-treatment with EGCG (0, 5, 10, 15, 20 and 30 μM) for 24 h. Panel (g) shows the synergistic effect of the pharmaceutical composition comprising baicalein and EGCG on TDP-43 misfolded aggregate reduction. Panels (h) and (i) show the synergistic effects of the pharmaceutical composition comprising baicalein and 17-AAG and the pharmaceutical composition comprising EGCG with 17-AAG on TDP-43 misfolded aggregate reduction at 7 hours and 24 hours, respectively. All data are presented as mean with SD.

FIG. 3 is an assembly of images illustrating baicalein restored TDP-43-mediated CFTR exon 9 skipping in an inherited VCP/p97 mutation cell-based model of ALS. Panel (a) contains in-vivo splicing analysis of TDP-43 in the presence of the VCP/p97 mutant R155H with or without baicalein. Exon 9 inclusion (+) and exclusion (−) bands are indicated. *: aberrant splicing product. Panel (b) contains in-vivo splicing analysis of TDP-43-mediated CFTR exon 9 skipping in cells treated with or without baicalein. Panel (c) illustrates in-vivo splicing analysis of the effect of baicalein on CFTR exon 9 skipping in the absence of TDP-43 overexpression. Panel (d) is a photograph of a western blot showing the expression of TDP-43 proteins with or without baicalein. 293T cells were treated with 0, 25, or 50 μM baicalein following separation into nuclear, cytosolic, and urea fractions. Arrowhead indicated TDP-43 polymers. Panel (e) is a photograph of a western blot showing TDP-43 proteins in VCP/p97 R155H-expressing cells with or without baicalein. Arrowhead indicated TDP-43 polymers.

FIG. 4 is an assembly of images illustrating Analysis of VCP/p97 ATPase activities in TDP-43 polymerization and TDP-43-mediated CFTR exon 9 skipping Panel (a) contains microphotographs illustrating Localization of TDP-43 in cells transfected with VCP/p97-WT or VCP/p97-QQ. Bars: 10 Panel (b) is a photograph of a western blot showing the level of TDP-43 polymers (arrowhead) in cells transfected with 0.1, 0.2, 0.5, 1.5, or 2.0 μg VCP/p97-wt. Panel (c) contains immunoblot analysis of the level of TDP-43 polymers (arrowhead) in cells transfected with 0.1, 0.2, 0.5, 1.5, or 2.0 μg VCP/p97-QQ. Panel (d) contains in-vivo splicing analysis of TDP-43 in the presence of VCP/p97 variants. Exon 9 inclusion (+) and exclusion (−) bands are indicated. *: aberrant splicing product. Panel (e) contains in-vivo splicing analysis of TDP-43 in VCP/p97-QQ-expressing cells with or without baicalein. Panel (f) illustrates the interactions between TDP-43 and VCP/p97 in vivo by cross-IP. The protein lysate harvested from His-VCP/p97 variant-expressing 293T cells was used for immunoprecipitation with anti-His antibodies and was further examined by immunoblotting with anti-TDP antibodies. Panel (g) contains in-viva splicing analysis of TDP-43 in the presence of the R361S mutant of TDP-43 and VCP/p97 variants. Panel (h) contains in-vivo splicing analysis of the R361S mutant of TDP-43 in VCP/p97-QQ-expressing cells with or without baicalein.

FIG. 5 is an assembly of images illustrating TDP-43 polymerization and TDP-43-mediated CFTR exon 9 skipping by HSPB1 (HSP27). Panel (a) illustrates immunoblot analysis of the level of TDP-43 polymers in cells transfected with 0, 100, or 200 pmol siRNA of HSPB1 (HSP27). Arrowhead indicated TDP-43 polymers. Panel (b) illustrates the alternative splicing analysis of TDP-43 in the presence of HSPB1 siRNA by an in vivo splicing assay. Exon 9 inclusion (+) and exclusion (−) bands are indicated. *: aberrant splicing product. Panel (c) contains immunoblot analysis of the level of TDP-43 polymers in cells transfected with 5 or 10 μg plasmid of GFP-HSPB1. Arrowhead indicated TDP-43 polymers. Panel (d) contains the in vivo alternative splicing assay of TDP-43 in cells expressing GFP-HSPB1.

FIG. 6 is an assembly of the images illustrating identification of nuclear TDP-43 complexes and polymers. Panel (a) contains schematic diagrams of immunoprecipitation-EM illustrating the isolation method as applied to cellular glutamine/asparagine-rich protein complexes. Panel (b) illustrates immunoprecipitation efficiency of TDP-43, CBP, and TIAR, with or without pre-fixation. These three proteins were purified from 293T cell lysates by following the modified protocol described in a using an anti-TDP-43, or TIAR, CBP antibody. The immunoprecipitates were further examined via immunoblotting with an anti-TDP-43, or TIAR, CBP antibody. Panel (c) contains microphotographs illustrating negatively stained immunoprecipitates using anti-TDP-43 antibodies. Bars: 20 nm. Arrowheads indicated representative high-magnification images of isolated TDP-43 complexes in d-f and h-j. Panel (d), (e) and (f) contains microphotographs illustrating three selected electron micrographs of the negatively stained structures of TDP-43 polymers isolated from the cell lysates following a pre-fixation processor. Helical polymer structure of TDP-43 isolated from cells. The selected image in f illustrated two branches of a TDP-43 polymer. Scale bars: 20 nm. Panel (g) contains microphotographs illustrating straight immunogold labeling of TDP-43 proteins in the nucleus of 293T cells. Scale bars: 100 nm. Panel (h), (i), and (j) contains microphotographs illustrating single spherical structures from different micrographs. Scale bars: 20 nm. Panel (k) contains electron micrographs of negatively stained structures of the fibrogranular network of TDP-43 isolated without pre-fixation. Scale bars: 100 nm. Panel (1) contains electron micrographs of negatively stained structures of the fibrogranular network of TDP-43 isolated with pre-fixation. Scale bars: 100 nm. Panel (m) contains immunofluorescence staining of endogenous TDP-43. Scale bars: 5 μm. Panel (n) illustrates the prion-like propensity of TDP-43 is required to form knobbed structure of fibrogranular network of TDP-43 by analysis of TDP-43-FL- and TDP-43-PLDA-expressing pattern. Arrowhead indicated fibrillar structures. Scale bars: 10 μm.

FIG. 7 is an assembly of images illustrating TDP-43 dysfunctions in Hutchinson-Gilford progeria syndrome. Panel (a) Immunostaining of TDP-43 and lamin A/C. Green: TDP-43; Red: lamin A/C, Scale bars: 5 μm. Arrowhead indicates colocalization of TDP-43 and lamin A. Panel (b) Localization of GFP-TDP-43-FL proteins in lamin A- and progeria-expressing cells. Arrowhead indicates cytosolic aggregates of TDP-43. Scale bars: 10 μm. Panel (c) Examination of TDP-43 alternative splicing ability in the presence of lamin A or progeria by an in vivo splicing assay. Exon 9 inclusion (+) and exclusion (−) bands are indicated. *: aberrant splicing product. Panel (d) Western blotting for the validation of TDP-43 polymers in progeria-expressing cells. Panel (e) Localization of TDP-43 in cells expressing progeria with or without baicalein. Bars: 10 Panel (f) The statistical analysis of baicalein rescue of TDP-43 nuclear localization is shown. All data are presented as the means with SD (n=3). *P<0.05 by t-test. Panel (g) Examination of TDP-43 alternative splicing ability in the presence of baicalein in cells expressing progeria by an in vivo splicing assay. Exon 9 inclusion (+) and exclusion (−) bands are indicated. *: aberrant splicing product. Panel (h) Immunostaining of nuclear shape in progeria-expressing cells with or without baicalein using anti-lamin A/C antibodies. Arrowhead in b indicates misshapen nuclei, and arrowhead in c shows rescued nuclei bars: 10 Panel (i) Western blotting for the validation of TDP-43 proteins in lamin A-expressing cells.

FIG. 8 is a schematic illustration of the model of small compounds in treating disease TDP-43 proteins. A proposed spatiotemporal organization for TDP-43-mediated exon skipping under normal physiology conditions. TDP-43 proteins reassembled into polymers carried out splicing functions at the nuclear fibrogranular network. At a prodromal or clinical disease stage that was caused by risk factors, such as ROS or inherited mutations, degraded TDP-43 C-terminus translocated into the cytosol, following aggregating pathological inclusions. Pharmacological intervention with baicalein disassembled pathological inclusions and rescued nuclear functions of TDP-43 by increasing the number of active TDP-43 polymers in the nucleus. Additionally, EGCG or 17-AAG could work effectively alone or in a synergistic manner with baicalein in reducing TDP-43 misfolded aggregates.

FIG. 9 is an assembly of images illustrating identification of the prion-like propensity of SMN. Panel (a) b-isox precipitates SMN proteins from mes23.5 cells. Panel (b) Analysis of the levels of the prion-like conformers of SMN and PFN1 in different subcellular fractions of 293T cells. Panel (c) Results for fractionated proteins from cell lysates treated with or without b-isox are shown. Panel (d) Schematics of SMN mutants. Panel (e) Identification of prion-like domain of SMN. Panel (f) Subcellular localization of SMN variants. Scale bar, 10 μm. Panel (g) Top panel, b-isox chemical binding analysis of the missense SMN mutants Y272C and G279V. Middle and bottom panels, solubility of Y272C and G279V mutants. Panel (h) Cellular expression patterns and localization of Y272C and G279V mutants. Panel (i) b-isox chemical binding analysis of SMNΔ7.

FIG. 10 is an assembly of images illustrating functional conversion of SMNΔ7 into full-length SMN by baicalein. Panel (a) Schematics of the proposed structural properties of full-length SMN, SMNΔ7 and SMA missenses mutants. Panel (b) Baicalein decreased the number of SMNA7 aggregates. All data are presented as the means with SD (n=3). *P<0.05 by t-test. Panel (c) Results of cell viability assays of SMNA7-expressing cells treated with 50 μM baicalein. All data are presented as the means with SD (n=3). *P<0.05 by t-test. Panel (d) Baicalein increased the number of SMNA7 cells with neurite-like structure. All data are presented as the means with SD (n=3). *P<0.05 by t-test. Panel (e) The physical interaction of SMNΔ7 with PFN1 was examined in the presence of baicalein. Panel (f) Baicalein attenuates the degradation of the SMNΔ7 protein. Panel (g) Effects of baicalein on the axon length of cultured NSC34 motor neurons. Baicalein (50 μM)- or mock-treated NSC34 were stained with the IMI-tubulin antibody (purple). SMNΔ7-transfected cells are shown by the red mCherry signal. Scale bar, 50 μm. Panel (h) Quantitation of the neurite length of SMNΔ7 transfected cells. Statistical comparisons were performed using two-tailed Student's t-tests. All data are presented as the means with SD (n=3). *** p<0.001. Panel (i) The therapeutic effects of baicalein on motor function in SMA mice. The SMA mice and heterozygous littermates were treated with daily intraperitoneal baicalein injections from birth, followed by motor functional analyses. Righting time (left panel), tube score (middle panel), and tilting score (right panel) of untreated (SMA, n=27; heterozygous, n=23) and baicalein-treated (SMA/TX, n=18; heterozygous/TX, n=27) SMA and heterozygous mice are shown. The motor function of SMA mice was significantly improved after baicalein treatment, particularly at postnatal day 6, and partially improved at postnatal day 8 (one-way ANOVA with LSD post hoc analysis). * p<0.05, ** p<0.01, *** p<0.001.

FIG. 11 is an assembly of images illustrating the effect of the level of prion-like domain on axon outgrowth from SMNA7-expressing motor neurons. Panel (a) Co-transfected NSC34 cells (indicated by arrows) were stained with the βII-tubulin antibody (purple). Scale bar, 50 μm. Panel (b) Quantitation of the neurite length of co-transfected cells. Statistical comparisons were performed using two-tailed Student's t-tests. All data are presented as the means with SD (n=3). *** p<0.001.

FIG. 12 is an assembly of images illustrating modeling of the prion-like conformer-based therapeutic strategy for treating SMA by correcting misfolded SMN proteins. Our study identifies a small-molecule structure corrector, baicalein for SMA that tackles the issue of insufficient levels of prion-like conformers through the pharmacological chaperone-induced “prion-like iso-conformers”.

FIG. 13 is an assembly of images illustrating in vivo structural templating of heterologous LC domains. Panel (a) A cellular image of GFP-Htt97Q. Scale bar: 10 μm. Panel (b) Solubility analysis of GFP-Htt97Q proteins. Panel (c) Templated folding and propagation of a cross-β conformer by GFP-Htt-97Q. Panel (d) Analysis of the levels of cross-0 conformers of TDP-43-FL, TDP-43-PLDΔ and TDP-43-F147/149L. B-isox binding analysis of cross-β polymers in cells expressing TDP-43-FL, TDP-43-PLDΔ and TDP-43-F147/149L (RD) proteins. Panel (e) B-isox binding analysis of cross-β polymers in cells expressing hTDP-43 FL and hTDPK136R proteins. Panel (f) Immunoprecipitation analysis of TDP interacting partners in cell lysates of hTDP-43 FL- and hTDPK136R-transfected cells. Panel (g) Statistical analysis of nuclear membrane localization of hTDP-43 FL and hTDPK136R. All data are presented as the mean with SD (n=5). *P<0.05 by t-test. Panel (h), Purified full-length TDP-43 and Lam B recombinant proteins (equivalent 3 μM monomer) were incubated in the absence (left) or the presence of b-isox (100 μM; middle) in assembly buffers at RT, followed by validation with electron microscopy. TDP-43 and Lam B recombinant proteins incubated separately for 1 hr and then mixed for 1 hr (right). Bar: 1 Panel (i) Analysis of the levels of endogenous cross-β conformer in different subcellular fractions of 293T cells. Panel (j) Calculation of the percentage of endogenous cross-β conformer of TDP-43 in Mes23.5 cells by western blotting.

FIG. 14 is an assembly of images illustrating analysis of cross-β conformer and templating in a cell-based model of ALS. Panel (a) B-isox binding analysis of prion-like proteins in cells expressing PFN1-FL and PFN1G118V proteins. Panel (b) Immunoprecipitation analysis of TDP interacting partners in cell extracts of PFN1-FL- and PFN1G118V-transfected cells. Panel (c) b-isox binding analysis of TDP-43 in VCP- and VCP R155H-transfected cells. Panel (d) Immunostaining of TDP-43 in VCP- and VCP R155H-transfected cells. Scale bar: 5 μm. Panel (e) A subcellular fractionation analysis of TDP-43 expression in VCP- and VCP A234E-transfected cells. Arrowhead indicates 90-kD TDP-43 dimers. Panel (f) A subcellular fractionation analysis of VCP and VCP R155H expression. Arrowhead indicates the fraction of chromatin-unbound VCP protein.

FIG. 15 is an assembly of images illustrating model of cross-β perpetuation. A novel type of β-sheet-rich domain is capable of structural replication by catalyzing the conversion of itself or other proteins and assembling into biopolymers, sequentially rebuilding the prion-like network to reshape cellular homeostasis, termed “cross-β-perpetuating”. Cross-β-perpetuating can be initiated by an increase in transformable LC proteins, RNAs, and posttranslation modification. This novel type of regulation dramatically reshapes cellular biochemistry by reorganizing the existing set of proteins.

FIG. 16 is an assembly of the images illustrating phenotypic characteristics of the misfolded p53 aggregates. Panel (a) and (b) contain microphotographs illustrating immunostaining of 293T cells with p53 antibodies (1C12). Arrows indicate the three types of aggregated p53 proteins. Bar: 10 μm. The statistical analysis of the size of p53 aggregates were shown in b. All the data are presented as the mean with SD (n=3). Panel (c) illustrates the finding that the effects of MG132 on p53 aggregation. Bars: 10 μM Panel (d) contains a flowchart illustrating the isolation of the p53 strains. Panel (e) contains selected images of the four p53 strains: p53 [L], p53 [S], p53 [P] and p53-NVA.Bars: 10 μm. Panel (f) contains immunostaining micrographs of the four p53 strains with an actin antibody. Bar: 10 μm. Panel (g) contains an analysis of p53 aggregate distribution during mitosis. Cells were double stained with p53 antibodies and DAPI. Bars: 10 μm. Panel (h) illustrates the findings for the detection of intracellular ROS levels in the four p53 strains. All the data are presented as the mean with SD (n=3). *P<0.05 by t-test.

FIG. 17 is an assembly of images illustrating the experimental findings of the studies of oncogenicity of p53 strains. Panel (a) contains graphs of the cell cycle distribution analysis of the p53 strains as verified by flow cytometry. Panel (b) contains bar graphs illustrating cell cycle doubling times for individual p53 strains. All the data are presented as the mean with SD (n=3). Panel (c) contains cell viability analysis of the four p53 strains treated with spermidine using an MTT assay. All the data are presented as the mean with SD (n=3). Panel (d) contains cell viability analysis of the four p53 strains treated with H₂O₂ using an MTT assay. All the data are presented as the mean with SD (n=3). Panel (e) contains western blot analysis of the expression profiles of the four p53 strains with specific antibodies relevant to cancer stemness and epigenetic regulation.

FIG. 18 illustrates p53 strain infectivity. Panel (a) contains microphotographs illustrating visible p53 aggregation induction in p53-NVA cells by incubation with lysates from p53 [L], [S], and [P] cells. Arrowheads indicate induced p53 aggregates. Bar: 20 μm. Panel (b) contains statistical analysis of p53 aggregate induction efficiency. All the data are presented as the mean with SD (n=3).

FIG. 19 is an assembly of experimental findings for illustrating that the reciprocal interplay of aggregation propensities between p53 and TDP-43. Panel (a) illustrates the localization of TDP-43 in four p53 strains. TDP-43 cytosolic foci was indicated by arrowhead. Bar: 10 μm. Panel (b) is a bar graph illustrating statistical analysis of cytosolic GFP-TDP-43FL aggregates in the p53 [S] and p53-NVA strains. All the data are presented as the mean with SD (n=3). Panel (c) is a bar graph illustrating statistical analysis of GFP-TDP-43IIPLD aggregates in the p53 [S] and p53-NVA strains. All the data are presented as the mean with SD (n=3). *P<0.05 by t-test. Panel (d) illustrates the western blots analysis showing of TDP-43 species in the four p53 strains by native PAGE. Panel (e) illustrates the in vivo alternative splicing analysis of the alternative splicing ability of TDP-43 in the p53 [S] and p53-NVA strains. Exon-9 inclusion (+) and exon-9 exclusion (−) bands are indicated. *: aberrant splicing product. Panel (f) contains bar graphs illustrating p53 aggregation in TDP-43-knockdown cells. Bar: 10 μm. Panel (g) contains bar graphs illustrating statistical analysis of the p53 aggregates in the TDP-43-knockdown cells. All the data are presented as the mean with SD (n=3). *P<0.05 by t-test. Panel (h) contains bar graphs illustrating clearance of p53 aggregates by overexpressing GFP-tagged TDP variants. Only FL and PLD efficiently clean p53 aggregates. p53 aggregates were detected by immunostaining using p53 1c12 antibody (n=5).

FIG. 20 is an assembly of images illustrating the effects of HSPB1 on p53 amyloid assembly. Panel (a) shows quantitative analysis of western blot data of HSPB1 proteins in the four p53 strains Panel (b) shows the mRNA expression levels of HSPB1, HSPB8 and HSP90 in the four p53 strains. Panel (c) contains a statistical analysis of p53 aggregate induction efficiency by HSPB1 knockdown in the p53-NVA strain. All the data are presented as the mean with SD (n=3). *P<0.05 by t-test. Panel (d) contains western blotting analysis of p53 solubility in HSPB1 knockdown cells. Arrowhead indicated insoluble proteins. Panel (e) contains a bar graph of p53 aggregates in four p53 strains over-expressing HSPB1. All the data are presented as the mean with SD (n=3).

FIG. 21 contains an analysis of misfolded p53 aggregates in cells expressing Wt p53 or the p53R280S mutant. Panel (a) shows p53 aggregates in cells overexpressing GFP-p53 or the GFP-p53R280S protein. 293T cells were transfected with a GFP-p53WT or the GFP-p53R280S plasmid and then stained with p53 antibodies. Bar: 10 μm. Panel (b) contains a bar graph of the cleaning efficiency of endogenous p53 aggregates in GFP-p53WT and GFP-p53R280S transfectants. All the data are presented as the mean with SD (n=3). *P<0.05 by t-test. Panel (c) shows the Western blotting analysis of the expression of CD133 and H3K27me3 in cells expressing GFP-p53 or GFP-p53R280S proteins. Panel (d) shows an analysis of p53 amyloid aggregates in cells treated with or without 25 μM baicalein. Selected images and the statistical analysis of p53 amyloids in cells treated with or without 25 μM baicalein are shown. All data are presented as the mean with SD (n=4). Bars: 10 Panel (e) illustrates suppression of the spontaneous formation of p53 aggregates by baicalein. All data are presented as the mean with SD (n=3). *P<0.05 by t-test. Bars: 10 μm.

FIG. 22 is an assembly of images illustrating identification of the prion-like propensity of Rb (Rb1). Panel Panel (a) Analysis of the levels of the prion-like conformers of Rb in different subcellular fractions of 293T cells. Panel (b) Schematics of Rb mutants and identification of prion-like domain of Rb. Panel (c-d) Analysis of the protein stability of Rb variants.

### DETAILED DESCRIPTION

Pharmaceutical compositions and methods for treating conformational diseases are described herein. The compositions comprising a combination of a heat shock protein modulator and a flavonoid or a composition comprising a combination of a heat shock protein modulator, a flavonoid and polyphenol compound. The polyphenol compound can be selected from a group comprising Apigenin, Catechin, Epicatechin, Kaempferol, 2,20-Dihydroxybenzophenon, 2,3,4,20,40-Pentahydroxyben-zophenone, Gossypetin, Quercetin, Morin, and Myricetin. When administered to cells, tissues or subjects, each of these various combinations synergistically reduces TDP-43 misfolded aggregate and/or TDP-43 degraded fragment.

Methods for stabilizing the biological forms of TDP-43 and SMN protein and/or restoring biological activity of TDP-43 and SMN protein, by administering a therapeutic agent, are also described herein.

In addition, methods are described herein for reducing TDP-43 and SMN misfolded aggregate or restoring biological form of TDP-43 and SMN proteins in a cell, a tissue or a subject by administering an effective amount of a therapeutic agent in the cell, the tissue or the subject to reduce the level of TDP-43 and SMN misfolded aggregates or increase active TDP-43 conformers. In one embodiment, the therapeutic agent is a flavonoid. In another embodiment, the therapeutic agent is a heat shock protein modulator. In another embodiment, the therapeutic agent is a polyphenol compound. In yet another embodiment, the therapeutic agent is the pharmaceutical composition described herein.

Also described are methods for altering the amount of TDP-43 polymers in a cell, a tissue or a subject by administering to the cell, the tissue or the subject a flavonoid in an effective amount to alter the level of TDP-43 polymers.

### Definitions

As employed above and throughout the disclosure, the following terms, unless otherwise indicated, shall be understood to have the following meanings.

As used herein, the singular forms “a,” “an,” and “the” include the plural reference unless the context clearly indicates otherwise.

As used herein, the term “about,” when referring to a measurable value such as an amount, a temporal duration, and the like, is meant to encompass variations of ±10%, from the specified value, as such variations are appropriate to the dosage of the therapeutic agent, unless otherwise specified. As used herein, the term “about,” when referring to a range, is meant to encompass variations of ±10% within the difference of the range from the specified value, as such variations are appropriate to the dosage of the therapeutic agent, unless otherwise specified.

An “effective amount,” as used herein, includes a dose of an agent that is sufficient to reduce the amount of TDP-43 degraded fragment or TDP-43 misfolded aggregate or p53 misfolded aggregate, or symptoms or signs of conformational disease.

The term “treating,” “treated,” or “treatment” as used herein includes preventative (e.g. prophylactic), palliative, and curative uses or results.

The term “reducing” or “reduce” includes slowing or stopping the formation of TDP-43, SMN or p53 misfolded aggregates or TDP-43 or SMN degraded fragments, or disassembling the TDP-43, SMN or p53 misfolded aggregates that have already been formed.

The term “prodrug” is a pharmacologically inactive compound that is converted into a pharmacologically active agent by a metabolic transformation.

The term “pharmaceutically acceptable salts” of an acidic therapeutic agent of the pharmaceutical composition are salts formed with bases, namely base addition salts such as alkali and alkaline earth metal salts, such as sodium, lithium, potassium, calcium, magnesium, as well as 4 ammonium salts, such as ammonium, trimethyl-ammonium, diethylammonium, and tris-(hydroxymethyl)-methyl-ammonium salts. Similarly, acid addition salts, such as of mineral acids, organic carboxylic and organic sulfonic acids, e.g., hydrochloric acid, methanesulfonic acid, maleic acid, are also possible provided to a basic therapeutic agent with a constitute such as pyridyl, as part of the structure.

The term “conformational disease” refers broadly to conditionals including, but not limited to, normal aging, premature aging, degradative conformational diseases, conformational disease (including amyloid aggregation conformational diseases and non-amyloid aggregation conformational diseases). The degradative conformational disease include but not limited to SMA, childhood cancer, retinoblastoma (RB), bladder cancer, breast cancer, osteogenic sarcoma and Rb (Rb1) deficient cancers. The amyloid aggregation conformational diseases include but not limited to Alzheimer's disease, Parkinson's disease, cancers with p53 aggregation, Down syndrome, or glaucoma. The non-amyloid aggregation conformational diseases include but not limited to TDP-43 proteinopathies (such as FTLD-U or ALS), hippocampal sclerosis or mixed proteinopathies.

The term “TDP-43 proteinopathy” as used herein refers broadly to a condition associated with the changes in one or more aspects of TDP-43 protein structure and/or function. TDP-43 proteinopathy can be characterized by deviations of the one or more aspects of TDP-43 protein structure and/or function from normal or baseline levels occurring in a population. These deviations can manifest themselves as abnormalities in structure of TDP-43 protein, such as amount of TDP-43 molecules having abnormal configurations, including TDP-43 degraded fragments and TDP-43 misfolded aggregates, or amount of various multimeric forms of TDP-43, including soluble and insoluble conformers. The deviations can also manifest themselves as abnormalities in cellular and tissue distribution of various molecular forms of TDP-43, deviations in functioning of TDP-43 protein, including loss of normal function, gain of toxic function or toxicity, or deviations in regulation of the proteins and cellular pathways related to TDP-43. The term “proteinopathy,” used in reference to TDP-43 or generally, can be used interchangeably with the terms “proteinopathy,” “misfolding disorder,” or “misfolding disease.” Examples of conditions that are currently considered TDP-43 proteinopathies are amyotrophic lateral sclerosis (ALS), frontotemporal lobar dementia with ubiquitin (FTDL-U), milder cognition impairments (MCI), Alzheimer's' disease (AD) and mixed pathology of neurodegeneration. It is to be understood that TDP-43 proteinopathies are not limited to the above conditions.

The term “prion-like LC proteins,” used in reference to TDP-43 or generally, can be used interchangeably with the terms “protein with cross-β perpetuating domain,” “low complexity protein,” “prion-like protein,” or “phase separated protein.”

The term “prion-like folding” as used herein refers broadly to a novel type of a β-sheet-rich structure is capable of structural replication by catalyzing self- or other protein conversion and sequentially forming physiological polymers. The prion-like domain variously described as a low-complexity (LC), cross-β propagation, cross-β perpetuating, aggregation-prone, prionogenic or liquid phase separation domain, transiently forms a cross-0 polymeric condensed phase to perform crucial biological processes, including membrane-less subcellular organs, pre-mRNA splicing, RNA polymerase II-dependent transcription, and heterochromatin relaxation. Additionally, b-isox, capture a group of prion-like LC protein, including TDP-43, FUS, hnRNPA1, TIA1, PFN1, Lamin B1, SMN, Rb and p53, acts as a specific chemical probe of the cross-β prion-like polymer of the LC domain.

The term “polymer,” used in reference to multivalent proteins or domains. The polymer can be analyzed by molecular weight using western blotting, morphology using electron microscopy, or droplet formation (such as, membrane less subcellular organ) using immunofluorescent staining, chemical precipitation using b-isox as probe.

The term “cross-β polymer,” used in reference to multivalent prion-like LC protein, can be used interchangeably with the terms “prion-like polymer” and recognized by b-isox.

The term “secondary aggregation-prone protein” used in reference to an aggregation-prone protein which compensates the role of a defective aggregation-prone protein. The secondary aggregation-prone protein can be expressed by a plasmid, or delivered by protein or lipid based nanoparticles, or Smart Mesoporous Silica Nanoparticles (see H. J. Liu et al “Smart Mesoporous Silica Nanoparticles for Protein Delivery” Nanomaterials 2019, 9(4), 511.).

The term “condition” can be used to refer to a medical or a clinical condition, meaning broadly a process occurring in a body or an organism and distinguished by certain symptoms and signs. The term condition can be used to refer to a disease or pathology, meaning broadly an abnormal disease or condition affecting a body or an organism. The term “condition” can also be used to denote a normal biological state or process.

The term “therapeutic intervention” as used herein refers broadly to actions taken that are expected to yield healing results, symptoms improvement or health restoration.

Forms of TDP-43 protein having different three-dimensional structure, meaning having differences in one or more of secondary, tertiary or quaternary structure, can be referred to as TDP-43 conformations, TDP-43 conformers, TDP-43 conformation variants, TDP-43 protein variants, TDP-43 folding variants, and by other related terms. It is to be understood that TDP-43 can have the same or different primary structure or amino acid sequence. TDP-43 conformers include TDP-43 monomers, oligomers or polymers, including soluble and insoluble monomers, oligomers or polymers. TDP-43 conformers include, but are not limited to the forms of TDP-43 found in vivo, including the forms associated with TDP-43 proteinopathies, the forms found in vitro, as well as the forms artificially generated. TDP-43 proteinopathies can be characterized by or associated with the amount of certain TDP-43 conformers in neural cells and tissues.

The term “amount” is used in this document to denote the quantity or distribution of something. In some embodiments, the present invention can utilize any of the foregoing information falling within the meaning of the term “amount” in relation to one or more proteins, as well as classes and subclasses of such proteins. Combination of such information on the amount of proteins can be referred to as “pattern.”

The term “subject” as used herein typically refers to a human or an animal having conformational disease or suspected of having conformational disease. It is to be understood that a subject can be subjects without known or suspected conformational disease, such as research subjects, are also included within the scope of the term “subject.”

The terms “heat shock protein” or “heat shock proteins,” respectively abbreviated as “HSP” and “HSPs,” refers to proteins involved in the “heat shock response,” a cellular response to increased temperatures or other stress factors that includes the transcriptional up-regulation of genes encoding heat shock proteins as part of the cell's internal protection and repair mechanism. HSPs, which are also called stress-proteins, are involved in various cellular reactions to stressful conditions, which include, but are not limited to, cold and oxygen deprivation. HSPs are also present and function in cells under normal conditions. Some HSPs are molecular chaperones that assist proteins in acquisition and maintenance of correct structure. For example, HSP chaperones can assist in protein folding and prevent aggregation of protein molecules. Other HSPs can shuttle proteins from one compartment to another inside the cell, and target misfolded proteins to proteases for degradation. Heat shock response is discussed, for example, in Richter et al., “The Heat Shock Response: Life on the Verge of Death,” Molecular Cell 40:253 (2010).

Agents that modulate heat shock protein activity or heat shock protein pathway in a cell, tissue or organism can be referred to as “heat shock protein modulators.” Heat shock protein modulators can activate or inhibit the function of an HSP or HSP pathway by various mechanisms. HSP modulators that decrease or inhibit the activity of a heat shock protein or pathway are referred to as HSP inhibitors. One example of a heat shock modulator is 17-N-allylamino-17-demethoxygeldanamycin (17-AAG), which is a derivative of the antibiotic geldanamycin. 17-AAG binds and inhibits the activity of HSP90 (heat shock protein 90), a protein chaperone that binds to signaling proteins, known as “client proteins.” 17-AAG is able to disrupt the HSP90-client protein complexes. HSP modulators that activate or increase the activity of a heat shock protein or pathway are referred to as HSP activators. One example of a heat shock protein modulator that is an activator is arimoclomol, which is known to induce expression of one or more molecular chaperone HSPs, such as HSP70 and HSP90.

The term “flavonoid” includes a flavone, which includes baicalein originally isolated from the roots of Scutellaria baicalensis. Baicalein is an inhibitor of CYP2C9, an enzyme of the cytochrome P450 system that metabolizes drugs in the body. The flavonoid includes baicalein and its derivatives.

Methods of Remodeling TDP-43 Aggregates and Stabilizing the Biological Form of TDP-43 Protein

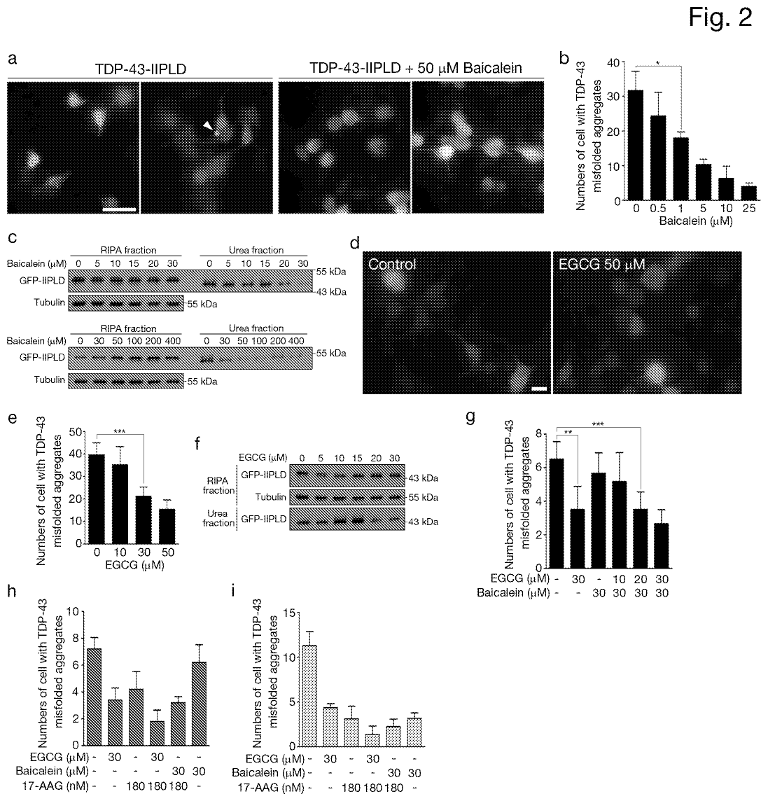

A neurodegenerative disease refers to diseases, such as TDP-43 proteinopathy, with proteins prone-to-aggregates. The Q/N-rich domain of TDP-43 could be functionally substituted with the yeast prion domain Sup35N, and has novel intrinsic property for cellular functions, including pre-mRNA splicing, subcellular localization, the exon skipping of CFTR, nuclear granular assembly and cellular folding stability. (Wang et al., “The self-interaction of native TDP-43 C terminus inhibits its degradation and contributes to early proteinopathies.” Nature Communication. 3:766 (2012) 2012). This Q/N rich domain of TDP-43 C terminus is also known as “prion-like domain or PLD.” However, in contrast to most known prions, the functional or misfolded aggregates of TDP-43 in vitro do not react with the amyloid-specific dye Congo red, indicating that the TDP-43 PLD may not be a prionogenic domain (Wang et al., Nature Communication. 2012). PLD participates in cellular folding in which the native TDP-43 C-terminus is stabilized, multiple TDP-43 proteins are interconnected to form TDP-43 functional aggregates in the nucleus and functional TDP-43 polymers is increased (see FIGS. 2, 6 and 7).

Inside the living body, the folded states of conformational disease proteins have been associated with aging and the pathogenesis of neurodegenerative diseases. These disease-causing proteins contain an intrinsic disordered domain that has high structural plasticity and structural polymorphisms and allows for switching in folding states with various biological and pathological factors, such as cellular binding, post-transcription modification, and ROS. The pathological effects on the folding states of this type of protein leads to misfolded aggregations and failure to maintain homeostasis and can cause neurodegeneration.

Under normal conditions, a prion-like nature engaged self-assembled TDP-43 proteins to cluster in the fibrogranular network in 3D nuclear space, where TDP-43 proteins carry out alternative splicing functions and become mRNA processing hubs. At the prodromal or clinical disease stage, pathological risk factors induced the functional folding of TDP-43 proteins to convert into a misfolded state, followed by ubiquitination, phosphorylation and aggregation in the cytosol. In the case of VCP/p97-associated TDP-43 proteinopathies, less TDP-43 immunoprecipitated by VCP/p97 and VCP/p97 R155H has been suggested in a spectrum of TDP-43 proteinopathies. Our studies herein further suggested that VCP/p97 R155H mutation or deficient in VCP/p97 ATPase activity interfered with the cellular localization and nuclear assembly of high-order TDP-43 polymers, sequentially disrupting the exon skipping ability of TDP-43. Additionally, sporadically or inherited FTLD/ALS-associated TDP-43 mutations (i.e., TDP-43 R361S) have also been found to accelerate pathogenesis. Accordingly, we found that the off-pathway compounds baicalein and EGCG disassembled insoluble non-amyloid pathological aggregates of TDP-43 into soluble fractions and effectively created synergistic effects with 17-AAG on reducing misfolded aggregates at 24 h post-treatment in vivo. Remarkably, baicalein not only disassembled TDP-43 fibers but also functionally corrected disease TDP-43 proteins into active TDP-43 polymers with the TDP-43-mediated exon skipping of CFTR. Applicants believe that redirecting the folding states of disease-causing proteins to an active state could simultaneously resolve several misfolded protein pathologies, including symptoms derived from gain- and loss-of-functions of disease proteins themselves, prion-like spreading and off-target effects. A single small-compound therapy treating multi-misfolded disease proteins of neurodegeneration would be safer than combination therapeutics for alleviating severe and complex pathologies of patients with concomitant mixed proteinopathies.

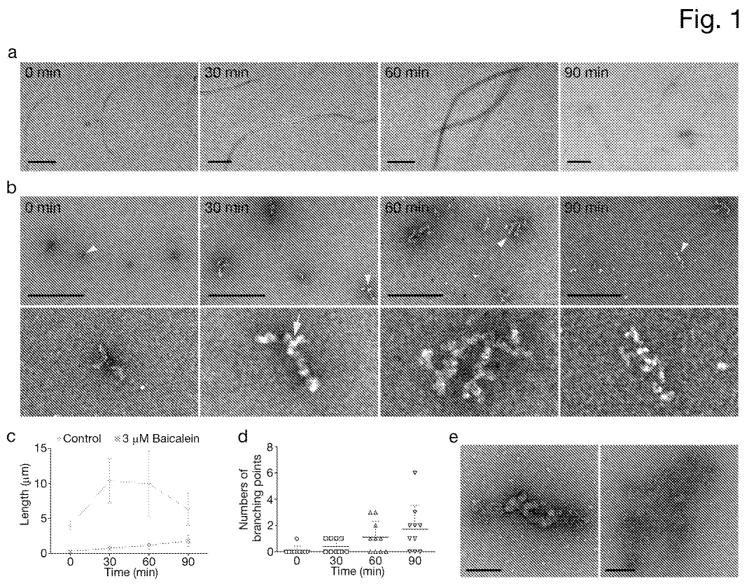

Methods of stabilizing the cellular folding of TDP-43 protein are provided herein by administering a therapeutic agent. In one embodiment, the therapeutic agent is a flavonoid, which stabilizes the cellular folding of TDP-43 proteins (see FIGS. 1, 2 and 3 for various embodiments of baicalein), whereby baicalein remodels TDP-43 fibers into polymers in vitro and there is more TDP-43 functional conformers in the nucleus. Additionally, isoxazole capture a group of prion-like protein, including TDP-43 suggested isoxazole potentially act as baicalein in therapeutic intervention of TDP-43 proteinopathies.

Methods for Reducing Insoluble TDP-43 Degraded Fragment and Misfolded TDP-43 Aggregates

Without being bound by any particular theory, it is believed that the loss of PLD in TDP-43 C-terminus by pathological cleavage, ALS-linked mutations or other unknown cellular factors causes disruption of cellular or prion-like folding of the TDP-43 C-terminus, and TDP-43 protein degrades and forms TDP-43 degraded fragments and/or TDP-43 misfolded aggregates. TDP-43 misfolded aggregates can lead to severe neuron loss and onset of TDP-43 proteinopathy.

The TDP-43 degraded fragment is soluble, about 22 to about 27 kDa (Neumann et al, “Ubiquitinated TDP-43 in Frontotemporal Lobar Degeneration and Amyotrophic Lateral Sclerosis. Science 314, 130 (2006)). The TDP-43 degraded fragments can lead to the formation of TDP-43 misfolded aggregates in the cytoplasm as shown in FIG. 2.

In various embodiments of the methods provided herein, the therapeutic agent is either a heat shock protein modulator, a polyphenol compound, a flavonoid or a combination, as described herein with reference to the pharmaceutical compositions provided, to reduce the level of insoluble TDP-43 degraded fragment or TDP-43 misfolded aggregate in a cell, a tissue or a subject. Reduction of the level of insoluble TDP-43 degraded fragment and/or TDP-43 misfolded fragment in a cell, a tissue or a subject can have a beneficial effect on a TDP-43 proteinopathy in a subject, such as, but not limited to, decreased risk or incidence of TDP-43 proteinopathy, attenuating or suppressing the progression of TDP-43 proteinopathy, suppression of neural degeneration, improvement of motor and/or neural functioning, reduction of symptoms and signs of TDP-43 proteinopathy, slowing down the progression of TDP-43 proteinopathy, and increasing the lifespan of the subject having TDP-43 proteinopathy.

The methods described herein are useful for reducing a detectable amount of a insoluble TDP-43 degraded fragment and/or TDP-43 misfolded aggregate, such as reduction of the amount of the TDP-43 degraded fragment and/or TDP-43 misfolded aggregate, degradation or disassembly of a TDP-43 degraded fragment and/or TDP-43 misfolded aggregate, such as disassembly of the TDP-43 misfolded aggregate, transition of TDP-43 degraded fragment to a functional TDP-43 protein associated with healthy cells, or changing the distribution or partitioning of a TDP-43 degraded fragment and/or TDP-43 misfolded aggregate within a cell or a tissue.

To perform the methods provided herein, a therapeutic agent selected from a heat shock protein modulator, a polyphenol compound, a flavonoid or a pharmaceutical composition described herein is administered to a cell, a tissue or a subject in an amount effective to reduce TDP-43 degraded fragment and/or TDP-43 misfolded aggregate in the cell, the tissue or the subject. The methods provided herein encompass therapeutic methods and uses, including methods of treating or attenuating TDP-43 proteinopathies, and prophylactic methods, including methods of preventing or reducing the probability of amount of TDP-43 proteinopathies in a subject. The methods provided herein also encompass research methods and uses, including in vitro and ex vivo methods of reducing TDP-43 degraded fragment of TDP-43 misfolded fragment in the cell, the tissue or the subject. Uses of a heat shock protein modulator, a polyphenol compound, a flavonoid or a pharmaceutical composition described herein for production of medicaments for reducing insoluble TDP-43 degraded fragment or TDP-43 misfolded fragment in the cell, tissue or subject are also encompassed by the embodiments of the methods described herein.

The methods provided herein reduce a variety of TDP-43 misfolded aggregates. In one embodiment, the TDP-43 misfolded aggregate is a misfolded aggregate from fusion of the degraded fragment of TDP-43 C-terminus, which mimic TDP-43 pathological fragments. In another embodiment, the TDP-43 misfolded aggregate is a misfolded aggregate from the full length TDP-43 protein.

The TDP-43 degraded fragment and TDP-43 misfolded aggregate are located in the cytoplasm of the affected cells and are “non-amyloid structure” as they do not react with the amyloid-specific Congo red.

Non-limiting examples of HSP modulators that can be administered to reduce TDP-43 degraded fragment or TDP-43 misfolded aggregate are 17-AAG, a pharmaceutically acceptable salt thereof, a derivative thereof, a prodrug thereof, or a structural analogue thereof. In one embodiment, the effective amount of 17-AAG s about 150 nM to about 400 nM.

Non-limiting examples of polyphenol compounds that can be administered to reduce TDP-43 degraded fragment or TDP-43 misfolded aggregate are EGCG, a pharmaceutically acceptable salt thereof, a derivative thereof, a prodrug thereof, or a structural analogue thereof.

Non-limiting examples of flavonoids that can be administered to reduce TDP-43 degraded fragment or TDP-43 misfolded aggregate are baicalein, a pharmaceutically acceptable salt thereof, a derivative thereof, a prodrug thereof, or a structural analogue thereof.

For administration according to the methods provided herein, heat shock protein modulators, polyphenol compounds, or flavonoid are administered alone or incorporated into suitable pharmaceutical compositions, alone or in combination, such as the pharmaceutical composition described herein.

Methods for Increasing Functional TDP-43 Polymers

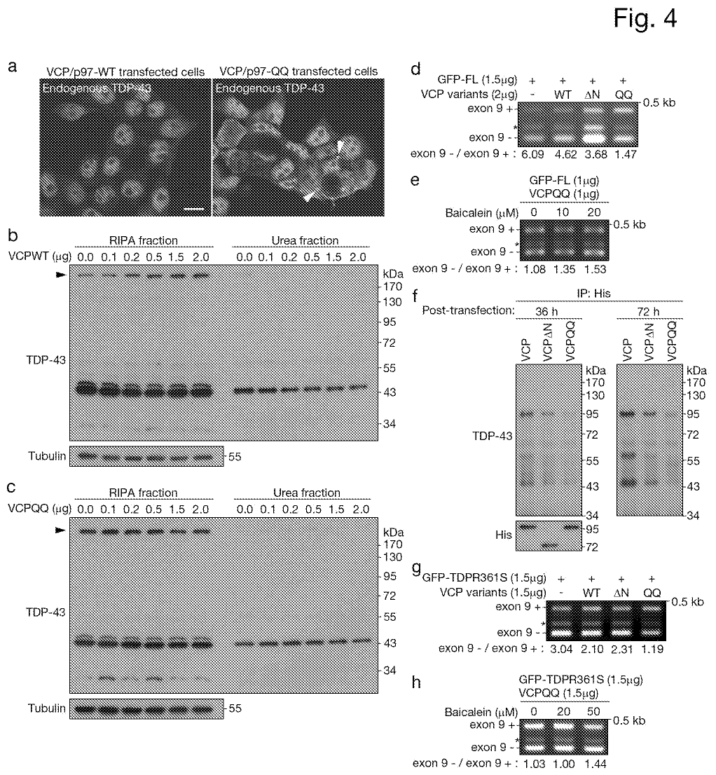

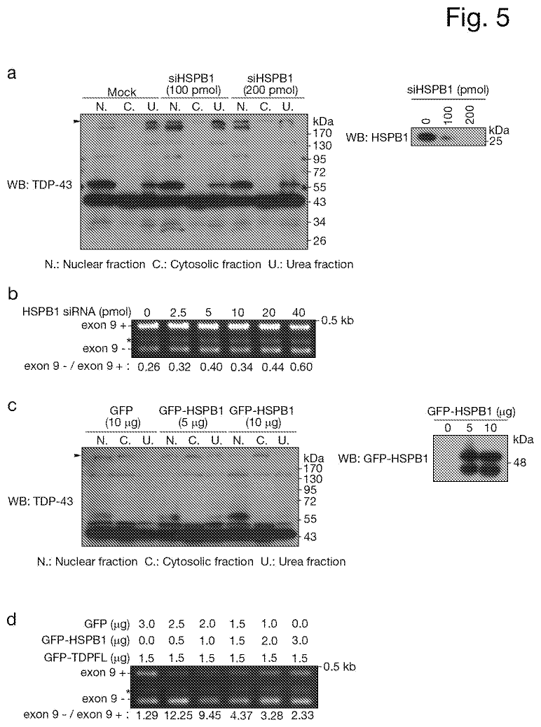

It was discovered by applicants that the ATPase activity of VCP/p97 was involved in the cellular localization of TDP-43 and the assembly of TDP-43 polymers (FIG. 4). Applicants also discovered that HSPB1 expression affected the assembly TDP-43 polymers tied with TDP-43-mediated exon skipping, which provided baicalein-independent evidence for the new species; i.e., nuclear TDP-43 polymers, which perform TDP-43-mediated exon skipping (FIG. 5). Accordingly, applicants discovered that the ATPase activity of VCP/p97 and HSPB1 are effective drug targets for influencing TDP-43 conformer conversions and increasing TDP-43 polymers and thus correction of TDP-43 proteinopathies.

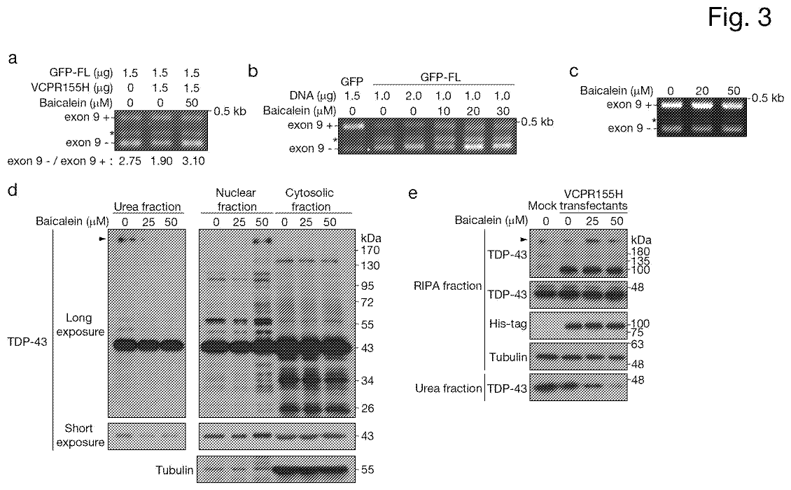

TDP-43 proteinopathies in FTLD/ALS with the VCP/97 mutation R155H have been characterized. The VCP/97 mutation R155H alters the functions of VCP/97, redistributes TDP-43 to the cytosol, and leads to form insoluble aggregates of TDP-43. A functional correlation between rescuing the TDP-43-mediated exon skipping of CFTR and the appearance of increased TDP-43 polymers was also observed in baicalein-treated VCP/p97 R155H cells (FIG. 3e)

Less TDP-43 immunoprecipitated by VCP/p97 has been suggested in a spectrum of TDP-43 proteinopathies, which implies that the interference of interactions between VCP/p97 and TDP-43 is a key step of pathogenesis in patients with sporadic or inherited TDP-43 proteinopathies. Significantly defective interactions of VCP/p97 and TDP-43 lead to assembly failures of TDP-43 polymers that can be corrected by baicalein.

Methods of using an modulator of HSPB1 or ATPase activity of VCP/p97 or an flavonoid, such as baicalein and its derivatives and structural analogues, in order to alter amount of one or more functional TDP-43 polymers in a cell, tissue or subject are provided herein. Examples of HSPB1 (also known as HSP27) modulator include siRNA against HSP27. In an exemplary embodiment, the siRNA against HSP27 is at least 90, 95 or 100% identical to SEQ ID NO:11, SEQ ID NO:12, SEQ ID NO: 13, SEQ ID NO: 14, SEQ ID NO: 15, SEQ ID NO: 16, SEQ ID NO:17 or SEQ ID NO:18.

Altering the amount of one or more TDP-43 conformer in a subject can have a beneficial effect on a TDP-43 proteinopathy in a subject, such as, but not limited to, decreased risk of incidence of TDP-43 proteinopathy, attenuating or suppressing the progression of TDP-43 proteinopathy, suppression of neural degeneration, improvement of motor and/or neural functioning, reduction of symptoms of a TDP-43 proteinopathy, slowing down of progression of a TDP-43 proteinopathy, and increasing in a lifespan of the subject.

To perform an embodiment of the methods provided herein, an protein expression modulator, such as HSPB1RNAi or VCP/p97 plasmid, or an flavonoid, such as baicalein and its derivatives and structural analogues, is administered to a cell, a tissue or a subject in an amount effective to alter one or more TDP-43 conformer in the cell, the tissue or the subject. The methods provided herein encompass therapeutic methods and uses, including methods of treating or attenuating TDP-43 proteinopathies, and prophylactic methods, including methods of preventing or reducing the probability of amount of TDP-43 proteinopathies in a subject. The methods also encompass research methods and uses, including in vitro and ex vivo methods of altering amount of one or more functional TDP-43 conformer in the cell, the tissue or the subject. Uses of HSP modulators for production of medicaments for altering amount of one or more functional TDP-43 conformer in the cell, tissue or subject are also encompassed by the embodiments of the methods described herein.

The methods provided herein include altering amount of a variety of TDP-43 conformers. One example of TDP-43 conformer is a soluble TDP-43 polymer, such as soluble TDP-43 polymer having a molecular weight of 200 kDa or more (“soluble TDP-43 conformer”).

Altering the amount of one or more TDP-43 conformer in a cell, a tissue or a subject according to the methods provided herein encompasses reduction of a detectable amount of a TDP-43 conformer, such as reduction in the amount of the 200 kDa or more TDP-43 conformers in cytosol, degradation or disassembly of a TDP-43 conformer, such as degradation of the insoluble TDP-43 conformer, transition of TDP-43 from one conformer to another conformer, such as transition from a TDP-43 conformer associated TDP-proteinopathy to a functional multimer associated with healthy cells, or changing the distribution or partitioning of a conformer within cell or tissue.

Some examples of a flavonoid that can be administered to alter the amount of one or more TDP-43 conformers are, baicalein and its derivatives and structural analogues.

For administration according to the methods provided herein, a flavonoid can be administered alone or incorporated into suitable pharmaceutical compositions as described herein.

Methods for Functional Interaction of TDP-43 and Lamin a as Druggable Pathways for Treating TDP-43 Proteinopathies and Premature Aging

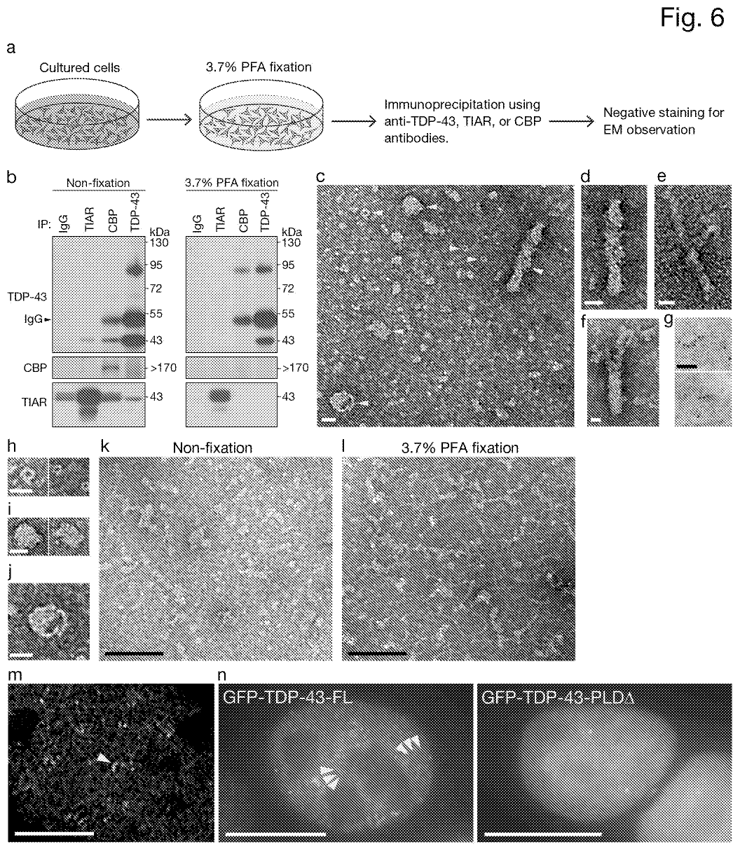

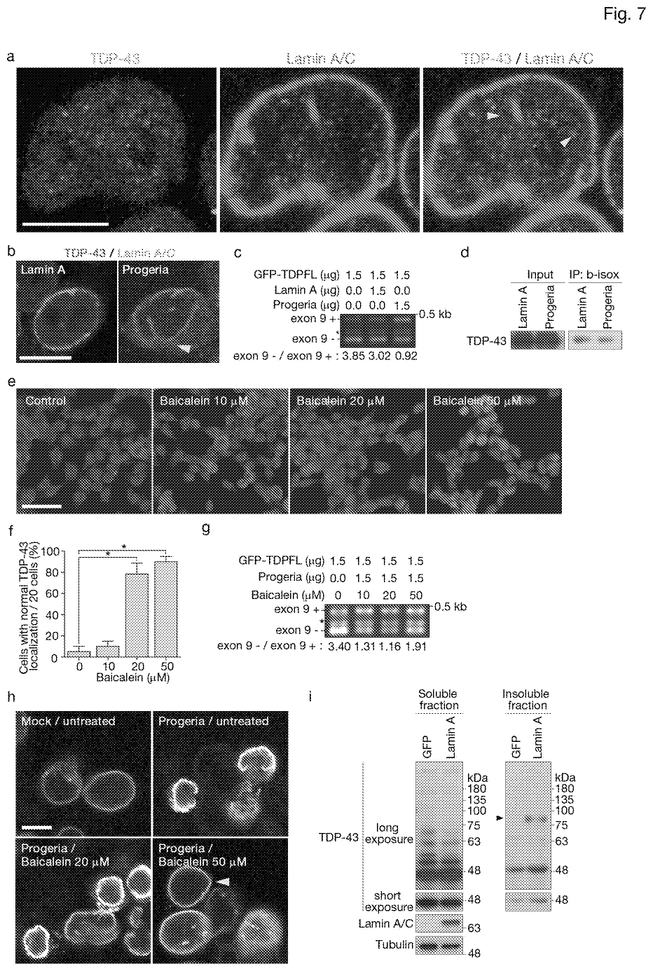

Applicants observed TDP-43 forms a fibrogranular network composed of branched, knobbed filaments that was associated with globular structures, similar to known fibrogranular ribonucleoprotein and that was connected with nuclear intermediate filament lamin A/C (see FIG. 6 and FIG. 7).

A defective lamin A mutant, progeria, causes a premature aging disorder, Hutchinson-Gilford progeria syndrome (HGPS). Applicants observed progeria mutation disturbed TDP-43 polymer assembly, resulting in the failure of TDP-43-mediated alternative splicing. TDP-43 dysfunctions likelihood leads to extensive changes in alternative splicing in patients with HGPS (FIG. 7). Significantly, the induction of cytosolic aggregation of TDP-43 by aging associated protein reveals a clue about aging dependency in TDP-43 proteinopathies.

Methods for Treating SMA by Increase the Level of the Prion-Like Folding of Aggregation-Prone Domain

Spinal muscular atrophy (SMA) causes the loss of motor neurons and progressive muscle weakness. In 95% of patients with SMA, both alleles of the survival motor neuron 1 (SMN1) gene are deleted, or the gene contains missense mutations.

It is noted that SMN has an intrinsic prion-like propensity, which drives homo- and hetero-cross-□ oligomerization of SMN to regulate Gems formation, SMN protein stability, and axonal outgrowth of motor neurons. Disease-causing missense mutations and exon 7 deletion (SMNA7) in the protein lead to a misfolded state and abolish functional prion-like interactions. These protein products appear to be unstable and rapidly degraded.

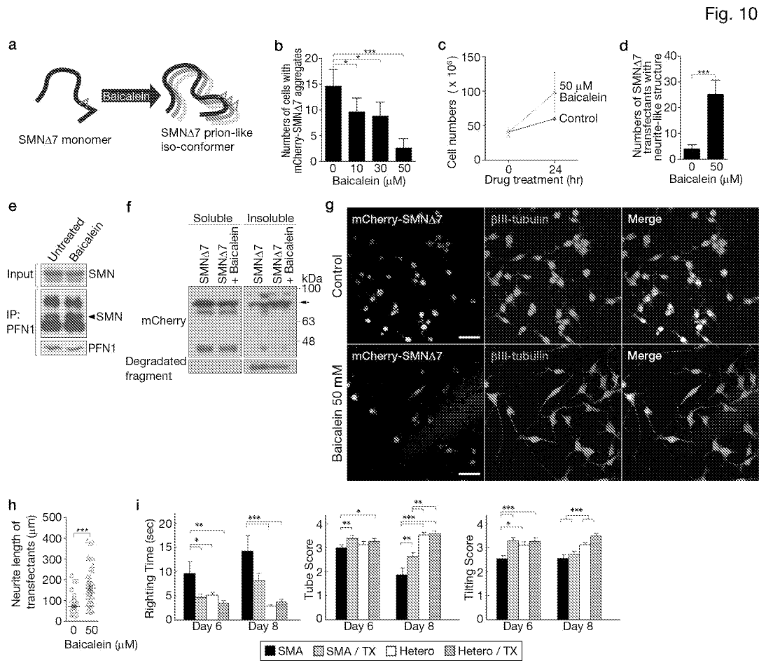

The forced reassembly of the prion-like conformer of SMNA7 by baicalein reduced degraded fragment, increased protein stability, prion-like interactions with other prion-like proteins, i.e., PFN1, axonal outgrowth and cell viability in cultured motor neurons expressing SMNA7 and improved motor functions in SMA mice (see FIG. 10).

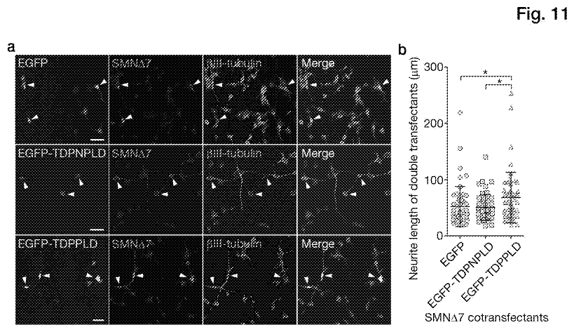

Restoration of the functional deficiency of SMNA7 in SMA mice was also achieved by overexpression of the prion-like domain of TDP-43 (see FIG. 11). These findings revealed an intrinsic molecular property of SMN, which is precisely linked to SMA, and offer a treatment to patients with SMA-causing mutations by simply restoring prion-like activity.

Methods for Reducing Misfolded p53 Aggregates

Misfolded p53 aggregates are commonly observed in malignant tumors, particularly in chemotherapy-treated tumors or highly metastatic cancers bearing p53 mutations. Thirty to forty percent of p53-associated cancer mutations affect the structure of the protein, resulting in increased propensity toward aggregation. Currently known p53 aggregate-positive cancer types include breast, colon, skin, ovarian and prostate cancers. p53 aggregates have been experimentally shown to form amyloid oligomers and fibrils similar to those identified in Alzheimer's disease, Parkinson's disease and prion diseases, which have beta-sheet registry amyloid structures due to binding to thioflavin T.

Methods for Drug Screening System of p53 Proteinopathies

It is unclear how toxic amyloids might induce tumorigenesis. In vivo experiments investigating this hypothesis are hindered by the lack of a simple system to analyze the effects of p53 amyloids on tumorigenesis because inducing p53 aggregation simultaneously inhibits p53 tumor suppressor activity, which can itself lead to carcinogenesis. Additionally, artificially adding p53 fibers to cell culture to induce the formation of cellular p53 aggregates causes cell death, which is at odds with clinical observations in which p53 aggregate-positive tumors show enhanced growth. Besides, none of downstream oncogenic effectors or pathways has been identified. Herein, we showed that spontaneous wild-type p53 (Wt p53) aggregation occurs in 293T cells, allowing us to study misfolded p53 aggregates in which p53 amyloids behave similarly as in clinical reports. We isolated and single clonally expanded four strains: three p53 amyloid strains-p53 [L] (long fibers), p53 [S] (short fibers), and p53 [P] (punctate aggregates), and the strain p53-NVA (no visible p53 ggregates). Individual p53 amyloid strain phenotypes are transmitted from parent strains to daughter cells. With these advantages, we investigated several important aspects of p53 aggregates, particularly their prion characteristics, oncogenicity, and their downstream effectors. Of note, unique four p53 strains isolated from 293T cells suggested that this popular cell line is actually a heterogeneous pool of cell types.

Applicants also found three p53 strain aggregates not only shared some prion features such as nucleation and horizontal transmission, but also influence cellular functions as other prion or prion-like proteins. However, only the p53 [P] strain was capable of infecting other cells.

Individual phenotypes had their own distinct features. A series of biochemistry, immunofluorescence and gene profiling analyses revealed distinct pathophysiologies for the three strains, such as a dramatic increase in ROS and loss of H3K27me3 in the p53 [L] strain. The expression of CD133, a cancer stem cell marker, was significantly increased in the p53 [S] and p53 [P] strains. Furthermore, only the p53 [P] strain, which exhibits a punctate phenotype, was capable of infecting cells, indicating a non-cell autonomous influence.

Methods for p53 Aggregates-Induced Misregulated Proteins, Anti-Amyloid Agents, Reducing HSPB1 Expression or Increasing p53 Expression as Druggable Targets of p53 Proteinopathies

Compared with the p53-NVA strain, p53 aggregation strains show accelerated cell growth, epithelial-to-mesenchymal transition (EMT) activation, increased cancer stemness. The identified common downstream effectors included proteins involved in hormonal-related concentration and EGFR pathways, as well as a group of epigenetic regulators, including H3K27me3, H3K27Ac and DNMT1, which are common pathological targets of p53 amyloids.

Applicants found lower HSPB1 expression, which is associated with p53 aggregation, was observed in the p53 strains and in HSPB1-knockdown cells.

Applicants also discovered an anti-amyloid agent and p53 plasmid overexpression effectively eliminates p53 aggregation in all strains within 24 hr and reduces cell viability and cancer stemness, providing a potential strategy for treating misfolded p53 aggregate-positive tumors.

Methods for Treating Amyloid-Positive Cancer and/or Cancer Stem Cells by Blocking Infectivity of Misfolded Disease Proteins

Following the addition of p53 amyloid extracts to non-amyloid contained cells, applicants observed p53 punctate can behave as an infectious entity.

p53 aggregates are capable of infecting other cells or tissue that suggesting prion-like transmission might occur in cancer progression. We thus proposed two potential therapeutic strategies for preventing and/or treating aggregation-positive tumors using vaccine or peptides. Antigen for anti-amyloid immunotherapy strategies is designed as “a chemical modified and/or mutated” protein fragments, peptides, derivatives, and variants thereof which can block nucleation and/or transmission of misfolded protein aggregation. Since these antigenic peptides can block nucleation, they can be further applied in therapy, as therapeutic peptides for treating amyloidogenic diseases. Current approaches of vaccine against neurodegenerative related aggregates are to reassemble pathological conformations antigens that may lead to inoculate seed of prion and cause proteinopathies. Indeed, evidence for human pathological Aβ and α-synuclein propagate like prions have recently been suggested (Jaunmuktane Z et al., 2015; Prusiner S B et al., 2015).

As p53 punctate can behave as an infectious entity and p53 autoantibodies were found in cancer patients. We proposed transmission of misfolded aggregates of p53 can induce immune response to generate specific autoantibody against p53 misfolded proteins in patients. Similar mechanism could occur in other amyloidogenic diseases such as neurodegenerative diseases or preeclampsia. Therefore, detecting proteopathic proteins' auto-antibody and/or misfolded aggregates of patients can be used to assess early proteniopathies of health people and patients with related diseases. The disease aggregates in plasma or CSF can be detected by aggregates-binding molecules including flavonoid(s), polyphenol(s) or polypeptide(s) such as human antibodies, immunoglobulin chain(s), fragments, derivatives, and variants thereof which binds to aggregates or oligomers of p53, TDP-43, amyloid oligomers, tau, Beta amyloid, IAPP, PrP^{SC}, Huntingtin, Calcitonin, Atrial natriuretic factor, Apolipoprotein A1, Serum amyloid A, Medin, Prolactin, Transthyretin, Lysozyme, Beta-2 microglobulin, Gelsolin, Keratoepithelin, Cystatin, Immunoglobulin light chain AL, S-IBM, carbonic anhydrase II, Retinoblastoma protein (pRb), Fus, and alpha-synuclein.

Methods for Treating Amyloid-Positive Cancer and/or Cancer Stem Cells by

Down-Regulation of Secondary Aggregation-Prone Proteins

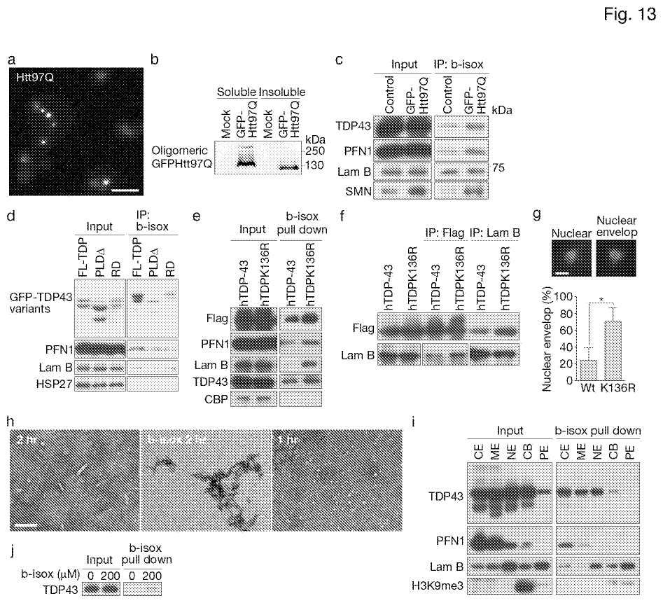

Applications found an interplay between TDP-43 and p53 in p53 amyloid-positive contents (FIG. 13). Herein, TDP-43 is the other prion-like protein in the context of p53 amyloid, thus we term TDP-43 as secondary aggregation-prone proteins. P53 is primary aggregation proteins.

Applicants also found a significant reduction in p53 amyloid fibers was found in cells transfected with TDP-43 siRNAs (FIGS. 13f and g).

Methods for Treating TDP-43 Proteinopathies by Interfering Amyloids

Applicants found the p53 amyloid strains can modulate the characteristics and cellular functions of other aggregation-prone proteins; for TDP-43, this included altered TDP-43 aggregation propensity that sequentially affected its ability to skip CFTR exon 9 (FIG. 13a-e).

Pharmaceutical Composition

Pharmaceutical compositions for stabilizing the cellular folding of TDP-43 protein, and reduction of TDP-43 degraded fragments and TDP-43 misfolded aggregates are provided herein. The pharmaceutical compositions provided herein are useful for reducing TDP-43 misfolded aggregate, preferably by advantageous synergistic effects of the combinations.

In one embodiment, the pharmaceutical composition includes a heat shock protein modulator, such as 17-AAG, a derivative thereof, a pharmaceutically acceptable salt thereof, or a prodrug thereof in combination with a polyphenol compound, such as EGCG, a derivative thereof, a pharmaceutically acceptable salt thereof, or a prodrug thereof. Optionally, the pharmaceutical composition further includes a flavonoid, such as baicalein, a derivative thereof, a pharmaceutically acceptable salt thereof, or a prodrug thereof.

In another embodiment, the pharmaceutical composition includes a heat shock protein modulator, such as 17-AAG, a derivative thereof, a pharmaceutically acceptable salt thereof, or a prodrug thereof in combination with a flavonoid, such as baicalein, a derivative thereof, a pharmaceutically acceptable salt thereof, or a prodrug thereof. Optionally, the pharmaceutical composition further includes a polyphenol compound, such as EGCG, a derivative thereof, a pharmaceutically acceptable salt thereof, or a prodrug thereof.

In another embodiment, the pharmaceutical composition includes a polyphenol compound, such as EGCG, a derivative thereof, a pharmaceutically acceptable salt thereof, or a prodrug thereof in combination with a flavonoid, such as baicalein, a derivative thereof, a pharmaceutically acceptable salt thereof, or a prodrug thereof. Optionally, the pharmaceutical composition further includes a heat shock protein modulator, such as 17-AAG, a derivative thereof, a pharmaceutically acceptable salt thereof, or a prodrug thereof.

The pharmaceutical compositions to be administered according to the methods of some embodiments provided herein can be readily formulated with, prepared with, or administered with, a pharmaceutically acceptable carrier. Such preparations may be prepared by various techniques. Such techniques include bringing into association active components (such as flavonoid, heat shock protein modulator or polyphenol compound) of the pharmaceutical compositions and an appropriate carrier. In one embodiment, pharmaceutical compositions are prepared by uniformly and intimately bringing into association active components of the pharmaceutical compositions with liquid carriers, with solid carriers, or with both. Liquid carriers include, but are not limited to, aqueous formulations, non-aqueous formulations, or both. Solid carriers include, but are not limited to, biological carriers, chemical carriers, or both.

The pharmaceutical compositions are administered in an aqueous suspension, an oil emulsion, water in oil emulsion and water-in-oil-in-water emulsion, and in carriers including, but not limited to, creams, gels, liposomes (neutral, anionic or cationic), lipid nanospheres or microspheres, neutral, anionic or cationic polymeric nanoparticles or microparticles, site-specific emulsions, long-residence emulsions, sticky-emulsions, micro-emulsions, nano-emulsions, microspheres, nanospheres, nanoparticles and minipumps, and with various natural or synthetic polymers that allow for sustained release of the pharmaceutical composition including anionic, neutral or cationic polysaccharides and anionic, neutral cationic polymers or copolymers, the minipumps or polymers being implanted in the vicinity of where composition delivery is required. Furthermore, the active components of the pharmaceutical compositions provided herein are useful with any one, or any combination of, carriers. These include, but are not limited to, anti-oxidants, buffers, and bacteriostatic agents, and optionally include suspending agents and thickening agents.

For administration in a non-aqueous carrier, active components of the pharmaceutical compositions provided herein are emulsified with a mineral oil or with a neutral oil such as, but not limited to, a diglyceride, a triglyceride, a phospholipid, a lipid, an oil and mixtures thereof, wherein the oil contains an appropriate mix of polyunsaturated and saturated fatty acids. Examples include, but are not limited to, soybean oil, canola oil, palm oil, olive oil and myglyol, wherein the number of fatty acid carbons is between 12 and 22 and wherein the fatty acids can be saturated or unsaturated. Optionally, charged lipid or phospholipid are suspended in the neutral oil. A suitable phospholipid is, but is not limited to, phosphatidylserine, which targets receptors on macrophages. The pharmaceutical compositions provided herein are optionally formulated in aqueous media or as emulsions using known techniques.

The pharmaceutical compositions provided herein may optionally include active agents described elsewhere herein, and, optionally, other therapeutic and/or prophylactic ingredients. The carrier and other therapeutic ingredients must be acceptable in the sense of being compatible with the other ingredients of the composition and not deleterious to the recipient thereof.

The pharmaceutical compositions are administered in an amount effective to reduce TDP-43 degraded fragment and/or TDP-43 misfolded aggregate, or to induce a therapeutic response in an animal, including a human. The dosage of the pharmaceutical composition administered will depend on the condition being treated, the particular formulation, and other clinical factors such as weight and condition of the recipient and route of administration. In one embodiment, the amount of the pharmaceutical composition administered corresponds from about 0.00001 mg/kg to about 100 mg/kg of an active component per dose. In another embodiment, the amount of the pharmaceutical composition administered corresponds to about 0.0001 mg/kg to about 50 mg/kg of the active component per dose. In a further embodiment, the amount of the pharmaceutical composition administered corresponds to about 0.001 mg/kg to about 10 mg/kg of the active component per dose. In another embodiment, the amount of the pharmaceutical composition administered corresponds to about 0.01 mg/kg to about 5 mg/kg of the active component per dose. In a further embodiment, the amount of the pharmaceutical composition administered corresponds to from about 0.1 mg/kg to about 1 mg/kg of the active component per dose.

Useful dosages of the pharmaceutical compositions provided herein are determined by comparing their in vitro activity, and in vivo activity in animal models. Methods for the extrapolation of effective dosages in mice, and other animals, to humans are known in the art; for example, see U.S. Pat. No. 4,938,949, which is incorporated by reference herein.

In accordance with the methods provided herein, the pharmaceutical compositions is delivered by any of a variety of routes including, but not limited to, injection (e.g., subcutaneous, intramuscular, intravenous, intra-arterial, intraperitoneal); continuous intravenous infusion; cutaneously; dermally; transdermally; orally (e.g., tablet, pill, liquid medicine, edible film strip); implanted osmotic pumps; suppository; or aerosol spray. Routes of administration include, but are not limited to, topical, intradermal, intrathecal, intralesional, intratumoral, intrabladder, intravaginal, intra-ocular, intrarectal, intrapulmonary, intraspinal, dermal, subdermal, intra-articular, placement within cavities of the body, nasal inhalation, pulmonary inhalation, impression into skin and electroporation.

Depending on the route of administration, the volume of the pharmaceutical composition provided herein in an acceptable carrier, per dose, is about 0.001 ml to about 100 ml. In one embodiment, the volume of a pharmaceutical composition in an acceptable carrier, per dose is about 0.01 ml to about 50 ml. In another embodiment, the volume of a pharmaceutical composition in an acceptable carrier, per dose, is about 0.1 ml to about 30 ml. A pharmaceutical composition may be administered in a single dose treatment or in multiple dose treatments, on a schedule, or over a period of time appropriate to the disease being treated, the condition of the recipient and the route of administration. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

In Vitro Drug Screening Methods for Conformational Diseases

The baicalein remodels existing TDP-43 aggregates into a soluble polymeric state in vitro and in vivo (FIG. 1 and FIG. 3). The soluble polymers of TDP-43 fulfill biological functions, ex. exon skipping of CFTR (FIG. 3 and FIG. 5).

Baicalein restores the bioactivity of misfolded TDP-43 proteins in multiple cell-based models of aging associated diseases (FIG. 3 and FIG. 7).

Baicalein-induced polymers of TDP-43 carry out TDP-43 nuclear functions. This suggests the polymerization of prion-like LC proteins can be used to screen therapeutic candidate for conformational diseases, to restore the bio-activities of misfolded prion-like disease proteins of conformational diseases.

Embodiments of the present invention are illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the invention. During the studies described in the following examples, conventional procedures were followed, unless otherwise stated. Some of the procedures are described below for illustrative purpose.

### Description of Materials and Methods Used in the Examples

The following materials and methods were used in the Examples described below.

Cell Culture and Drug Treatment: 293T cells were used throughout the experimental studies. 293T cells were grown in Dulbecco's modified Eagle's medium (DMEM)/F12, which was supplemented with 10% fetal bovine serum, 1% penicillin/streptomycin and 1% L-glutamate. Primary cultures of rat hippocampal neurons were prepared from embryonic day 17 rat embryos as previously described by (Wang et al, TDP-43, the signature protein of FTLD-U, is a neuronal activity-responsive factor. J. Neurochem. 105, 79T-806 (2008)). All procedures for handling the rat were carried out in accordance with the guidelines approved by the Institutional Animal Care and Utilization Committee, Academia Sinica. Hippocampal cells were plated on poly-1-lysine-coated coverslides at low density (10,000 cells per cm2) and cultured in the neurobasal medium/B27 (Invitrogen). The 293T cells were transfected using the calcium phosphate protocol according to the manufacturer's instructions and as previously described by Wang et al. (Wang et al., ProcNatlAcadSci USA. 99, 13583-13588 (2002). All of the plasmid constructs were described in Wang et al., Nature Communication, 2012). 293T cells (1×10⁵) were seeded in each well of a six-well plate and incubated overnight in a 37° C. incubator with 5% CO₂. To determine the effects of off-pathway stabilizers on the reduction of pathological-like aggregates, the cells were treated with baicalein and EGCG for 12, 24, or 48 h with the indicated concentrations (25 or 50 μM). To determine the synergistic effects of baicalein, EGCG, and 180 nM of 17-AAG on the reduction of pathological-like aggregates, cells were treated with the combination formula for 24 h post transfection with GFP-TDP-43-IIP plasmids.

Reagents and Antibodies: Baicalein and EGCG were obtained from Sigma. 17-AAG was purchased from Sigma and dissolved in dimethyl sulfoxide (DMSO). Primary antibodies against HSPB1 were purchased from Cell Signaling Technology (Beverly, Mass.). The primary antibody against lamin A/C was purchased from Millipore Inc. The primary antibody against U1 snRNP C was purchased from Sigma. Primary antibodies against DNMT1, eIF4A1, p-EGFR, and HIF1-α were purchased from Cell Signaling.com. The primary antibody against CD133 was purchased from abcam.com.

Isolation of Single p53 Strains by Limiting Dilution: 293T cells were diluted in Dulbecco's modified Eagle's medium (DMEM)/F12, which was supplemented with 10% fetal bovine serum, 1% penicillin/streptomycin to a final concentration of 1 cells/100 μl. Each well in a 96-well plate was seeded with 100 μl of cell suspension and cultured for 2 weeks. Only wells containing single colonies were further expanded.

Solubility Assay of TDP-43 and p53: Cells with or without transfection of TDP-43-FL or TDP-43-Q303P were lysed with RIPA buffer (50 mM Tris, 150 mM NaCl, 1 mM EDTA, 1% NP-40, pH 7.4), and the lysate was further fractionated by centrifugation at 16,000 g for 5 min at 4° C. The insoluble pellets were then dissolved in 8 M urea/50 mM Tris (pH 8.0). The proteins were identified by Western blotting using a polyclonal TDP-43 antibody or a monoclonal p53 antibody.

In vitro Analysis of TDP-43 Fiber Formation: Three-micromolar full-length TDP-43 recombinant protein (GenWay) was incubated with 3 μM baicalein in an assembly buffer. The reactions were performed for 30 min, 60 min, and 90 min with agitation at RT. The resulting samples were stained by 4% uranyl acetate for 1 min. EM analysis was performed with a FEI Tecnai G2 Spirit TWIN transmission electron microscope.

Infection assay. p53 [L], [S], or [P] cells were rinsed with PBS and resuspended in lml H₂O. After centrifugation, the supernatants and resuspended pellets were added to plated p53-NVA recipient cells. Twenty-four hours after exposure, cells were rinsed with PBS and fixed for immunostaining with using a p53-specific antibody (1C12; Cell Signaling #2524) antibody.

siRNAs and Transfections: siRNAs against HSPB1 (HSP27) and TDP-43 were purchased from Santa Cruz (SC-29350) and Dharmacon, respectively. For the overexpression and knockdown experiments, individual plasmids (3 μg) or siRNAs (25 or 60 pmol) were transiently transfected into 293T cells using Lipofectamine 2000 (Invitrogen), according to manufacturer's guidelines.

Plasmids: HSPB1 and p53R280S were individually amplified from human cDNA by PCR using primer sets HSPB1 and R280S. The resulting fragments were further cloned into pEGFP-N3 (Clontech, Mountain View, Calif., USA). GFP-p53 was generated by site-directed mutagenesis using GFP-P53R280S as the template. Site-directed mutagenesis was performed following the standard protocol using PfuUltra II HS Fusion DNA Polymerase (Agilent) and primer set S280R.

Immunohistochemistry: Fluorescence staining was performed as described previously (Wang et al., 2012). Cells were transfected with or without plasmids or siRNAs and grown for 24 to 48 hr. Cells were fixed with 3.7% paraformaldehyde in PBS at RT for 15 min and then permeabilized with 0.1% Triton X-100. The fixed cells were incubated with primary antibodies at RT for 2 hr followed by incubation with Cy2- or Cy3-labeled secondary antibodies (Molecular Probes). Slides were mounted using Vectashield DAPI H-1200 (Vector). Cellular fluorescence images were collected using an LSM710 META laser-scanning confocal microscope (Zeiss).

Alternative Splicing Assay: TDP-43-mediated CFTR exon 9 skipping assays were performed as previously described. Briefly, cells were cotransfected with TDP-43 plasmid, hCF-(TG)₁₃(T)₅ minigenes and the indicated plasmids, including VCP/p97 wt, VCP/p97 QQ, HSPB1, lamin A or progeria, or HSPB1 siRNA. Total RNA of transfectanted cells was isolated by TRIzol reagent (Invitrogen), and RT-PCR was carried out by Superscript III (Invitrogen) using specific primers to amplify exons 8-10 of CFTR according to the manufacturer's protocol. The relative amounts of cDNA were validated on 1.3% agarose gels.

Statistical Analysis: The statistical significance was calculated by analysis of variance (t-test). The difference between groups was considered to be significant if the P value was <0.05.

Mice: A mouse model of SMA was produced by deleting exon 7 of the Smn gene and knock-in of the human SMN2 gene (Smn−/−SMN2+/−). We were able to generate a variant that presents a more severe disease symptomatology through back-crossing to obtain a more homogenous genetic background. This model of severe SMA harbors two copies of the SMN2 transgene (Smn−/−SMN2+/−). The mouse model of SMA was generated through crossing the heterozygous knockout mice (Smn+/−SMN2−/−) with the homozygous knockout mice carrying two copies of the SMN2 transgene (Smn−/−SMN2+/+). SMA mice were subjected to daily intraperitoneal injections of baicalein (40 mg/kg/d in alcohol) or alcohol alone (control) from birth and then subjected to motor function tests and survival analysis. Three behavioral tests, the turnover test, tube test and negative geotaxis test, were conducted to evaluate the motor function of mice with SMA (Smn−/−SMN2+/−) and heterozygous littermates (Smn+/−SMN2+/−), as previously described. In the turnover test, the time required for a mouse to right itself and place all four paws on the ground from a prone position was recorded (the cutoff time was 60 s). The tube test was used to determine hind limb strength according to the posture of the hind limbs and the tail; scores ranged from 0 (the worst) to 4 (the best). In the negative geotaxis test, mice were placed on a 45° incline with their head pointing downward. The responses (turning and climbing) were scored from 0 (the worst) to 4 (the best).

B-isox Precipitation: The 293T cells were harvested and lysed with RIPA buffer. The protein concentration was adjusted to 1 to 10 mg/mL, and 10 mM biotinylated isoxazole was added to the cell lysate to a final concentration of 100 to 200 □M. The mixtures were then incubated at 4° C. for 60 min, centrifuged at 15000 rpm for 15 min at 4° C., and the supernatant was discarded. The entire reaction volumes were subjected to SDS-PAGE and western blotting.

### EXAMPLES

### Example 1: Baicalein, an Off-Amyloid Pathway Compound, Remodels Natively Unfolded Monomers and Misfolded TDP-43 Fibers into TDP-43 Polymers In Vitro

Functional substitution of the non-amyloid prion-like domain of TDP-43 with the amyloid prion domain of sup35 revealed a potential common structure for treating TDP-43 proteinopathies with off-amyloid stabilizers. To examine our hypothesis, we selected several known off-amyloid pathway compounds and validated their effects on the disassembly of TDP-43 misfolded aggregates. We incubated purified TDP-43 recombinant proteins with or without these off-pathway compounds (equimolar concentration) in assembling buffers at RT for 0, 30, 60, and 90 min with agitation. The effect of the off-pathway compounds was examined by negative staining electron microscopy. In the absence of these compounds, the length of TDP-43 fibers gradually increased from 3 to 10 μm (FIG. 1a). In the presence of baicalein, TDP-43 fibers, oligomers or natively unfolded monomers were efficiently remodeled into ordered TDP-43 polymers, where TDP-43 proteins resembled a globular structure (approximately 30 nm long) along a string in a time-dependent manner (FIG. 1b). Arrowheads indicated representative high-magnification images of TDP-43 polymers. The baicalein-inducing TDP-43 polymers were 0.15-0.9 μm long and considerably shorter than the TDP-43 fibers. The length of the TDP-43 fibers and TDP-43 polymers at different time points were analyzed (FIG. 1c). Notably, unlike tubular structures such as protofibers, these baicalein-inducing TDP-43 polymers had a highly branched structure, and the branches gradually increased to form ordered macrocomplexes with increasing reaction times (FIG. 1d). One of branching points was indicated by arrow in the lower panel of FIG. 1b. FIG. 1e show two selected images of polymerizing TDP-43 polymers. These results indicated that baicalein directly bound to and disassembled TDP-43 fibers and then transformed the aggregation state into TDP-43 polymers.

### Example 2: Baicalein, EGCG and 17-AAG Disassembles Pathological TDP-43 Inclusions In Vivo

Next, we validated the effects of baicalein on the disassembly of TDP-43 misfolded aggregates in vivo. GFP-TDP-43 pathological-like (GFP-TDP-43-IIPLD) inclusion-expressing 293T cells were treated with or without 50 μM baicalein for 12 h following microscopic analysis and Western blotting validation. As shown in FIG. 2a, baicalein inhibited the assembly of TDP-43-IIPLD proteinaceous nucleating particles (Arrowhead indicated TDP-43-IIPLD aggregates). A statistical analysis showed a dosage-dependent reduction of TDP-43-IIPLD aggregates by baicalein (FIG. 2b). In addition, Western blotting showed a reduction of insoluble TDP-43-IIPLD proteins in baicalein-treated cells. (FIG. 2c). Cells treated with a high dosage of baicalein (30-400 μM) had a significant compensatory increase in soluble fractions, suggesting that baicalein disassembled pathological TDP-43 aggregates instead of promoting degradation (FIG. 2c). Similarly, EGCG, the other off-pathway stabilizer, was also shown to reduce TDP-43 proteinaceous nucleating particles and increase soluble TDP-43-IIPLD proteins (FIGS. 2d, 2e and 2f). We found that two off-pathway compounds, baicalein and EGCG, disassemble the pathological-like aggregates of TDP-43 and redirect them into soluble fractions in cell-based disease models (FIG. 2a-f).

To further investigate potential therapies with a lower dosage and combination therapy with effective anti-aggregating compounds, we validated the synergistic effects of off-TDP-43 aggregate compounds, including baicalein, EGCG and 17-AAG, following treatments at 7 or 24 h (FIGS. 2g, 2h and 2i). We found baicalein and EGCG or 17-AAG exhibited synergistic effects in reducing TDP-43-IIPLD misfolded aggregates at 7 and 24 h post-treatment (FIGS. 2h and 2i).

### Example 3: Baicalein Rescued TDP-43 Dysfunctions in VCP97 Inherited Mutation Cell-Based Models of FTLD-U and ALS

We then tested whether baicalein can functionally rescue disease TDP-43 proteins. TDP-43 proteinopathies in FTLD/ALS with the VCP/97 mutation R155H have been characterized. The VCP/97 mutation R155H alters the functions of VCP/97, redistributes TDP-43 to the cytosol, and leads to form-insoluble aggregates of TDP-43. Because functional TDP-43 proteins are capable of promoting CFTR exon 9 skipping, we thus used an in vivo splicing assay to validate the folding state of cellular TDP-43 proteins in present of baicalein (FIG. 3a). Significantly, in cells co-transfected with TDP-43 and the VCP/p97 R155H mutant, TDP-43 failed to promote CFTR exon 9 skipping; however, this failure was corrected in the presence of baicalein (FIG. 3a). Cells treated with only baicalein showed an enhanced ability to promote the TDP-43-mediated CFTR exon 9 skipping in a dose-dependent manner (FIG. 3b). Without co-overexpressing TDP-43 proteins, baicalein had no effect on the exon skipping of CFTR (FIG. 3c), suggesting that the effect of baicalein on promoting the exon skipping of CFTR occurs through TDP-43 proteins. These results demonstrated that baicalein functionally corrected TDP-43 disease proteins in a VCP/97 mutation-induced disease model. We further investigated the pharmacological action of baicalein by analyzing the TDP-43 protein species of baicalein-treated cells because baicalein remodels TDP-43 fibers and natively unfolds TDP-43 monomers into polymers in vitro, as shown in FIG. 1. Indeed, we found that TDP-43 polymers significantly reduced the insoluble urea fractions in baicalein-treated cells but yielded a compensatory increase in the nuclear fraction (FIG. 3d, shown by arrowhead). A functional correlation between rescuing the TDP-43-mediated exon skipping of CFTR and the appearance of increased TDP-43 polymers was also observed in baicalein-treated VCP/p97 R155H cells (FIG. 3e). These results demonstrated that the ability of TDP-43 to promote the exon skipping of CFTR is associated with soluble TDP-43 polymers. Unfortunately, EGCG increased the level of the 130 kD TDP-43 instead of the high-order TDP-43 polymers that were insufficient to correct TDP-43 dysfunction on mRNA processing. This result implied differences in the pharmacological actions of baicalein and EGCG.

### Example 4: The ATPase Activity of VCP/p97 was Involved in the Assembly of TDP-43 Polymers and TDP-43-Mediated Exon Skipping

We further investigated whether the ATPase activity of VCP/p97 was involved in the cellular localization of TDP-43 and the assembly of TDP-43 polymers. We analyzed TDP-43 protein localization and TDP-43 polymers in 293T cells transfected with wild-type VCP/p97 or the ATPase-deficient VCP/p97 variant, VCP/p97-QQ (FIGS. 4a and 4b). We found TDP-43 formed cytosolic aggregates in cells expressing VCP/p97-QQ (FIG. 4a, arrowhead). TDP-43 polymers exhibited a gradually increase in cells transfected with VCP/p97-wt in a dose-dependent manner (FIG. 4b, arrowhead); conversely, gradually decreasing TDP-43 polymers were observed in VCP/p97-QQ-expressing cells (FIG. 4c, arrowhead). In vivo splicing assay further revealed that VCP/p97-QQ suppressed the TDP-43-mediated CFTR exon 9 skipping (FIG. 4d). Next, we tested whether baicalein could also rescue the VCP/97-QQ-induced inability of TDP-43 for CFTR exon 9 skipping and found that baicalein increased CFTR exon 9 skipping in VCP/p97-QQ-expressing cells (FIG. 4e). Cross-IP examination revealed that VCP/p97 physically interacted with TDP-43, which was consistent with studies by Gitcho et al. (FIG. 4f). A lower co-immunoprecipitation efficiency of TDP-43 with VCP/p97-QQ was observed compared to VCP/p97-wt, which suggested that VCP/p97ATPase activities functioning in interactions of TDP-43 and VCP/p97 to regulate the formation of high-order TDP-43 polymers. Of note, less TDP-43 immunoprecipitated by VCP/p97 has been suggested in a spectrum of TDP-43 proteinopathies, which implies that the interference of interactions between VCP/p97 and TDP-43 is a key step of pathogenesis in patients with sporadic or inherited TDP-43 proteinopathies. Significantly defective interactions of VCP/p97 and TDP-43 lead to assembly failures of TDP-43 polymers that can be corrected by baicalein.

Additionally, to examine whether baicalein could functionally correct disease-associated TDP-43 mutants, we designed a cell-based disease model of TDP-43 using the co-expression of FTLD/ALS-linked sporadic mutation TDP-43 R361S and VCP/p97-QQ. The reduced association of VCP/p97 and TDP-43 R361S and declined exon skipping capability of TDP-43 via VCP/p97-QQ overexpression were designed to simulate pathological conditions. We found that baicalein rescued dysfunction caused by the FTLD/ALS-linked sporadic mutation R361S of TDP-43 and a proline substitution mutant GFP-TDP-43-Q303P, which partially lost prion-like assembly, in cells expressing VCP/p97-QQ (FIG. 4g, 4h). These results implied that baicalein rescues not only misfolded wtTDP-43, but also inherited TDP-43 mutants.

### Example 5: The Loss of HSPB1 Increases Nuclear TDP-43 Polymers, Activating CFTR Exon 9 Skipping

We further investigated other cellular factors regulating the assembly of TDP-43 polymers. HSPB1, a potential regulator of TDP-43 functional aggregates, affects the assembly of high-order TDP-43 oligomers in the cytosol under oxidative stress and physically interacts with TDP-43. A deficiency of HSPB1 in 293T cells by transfected HSPB1 siRNA increased nuclear TDP-43 polymers in the nucleus (FIG. 5a). High-efficiency HSPB1 knockdown was confirmed (FIG. 5a). Furthermore, an in vivo splicing assay revealed a corresponding increase in CFTR exon 9 skipping in HSPB1 knockdown cells (FIG. 5b). Conversely, overexpression of HSPB1 increased TDP-43 polymers in cytosol and reduced CFTR exon 9 skipping (FIGS. 5c and 5d). HSPB1 expression affected the assembly TDP-43 polymers tied with TDP-43-mediated exon skipping, which provided baicalein-independent evidence for the new species; i.e., nuclear TDP-43 polymers, which perform TDP-43-mediated exon skipping.

### Example 6: Ultrastructure of TDP-43 Nuclear Complexes

To characterize the cellular configuration of TDP-43 nuclear complexes, particularly polymeric structures, we developed a modified method involving fixing cells prior to immunoprecipitation, followed by negative staining for electron microscopy. The experimental process for the purification of cellular TDP-43-containing complexes is illustrated in FIG. 6a. To preserve their structural integrity, the cells were partially fixed with 3.7% paraformaldehyde for 10 min prior to harvesting. We then fractionated cells and isolated TDP-43-, TIAR-, and CBP-associated cellular complexes through the immunoprecipitation of antibodies targeting these proteins from cellular nuclear extracts. Western blotting confirmed the successful purification of TDP-43 protein complexes (FIG. 6b). TIAR and CBP proteins were used as a control for specific isolation (FIG. 6b). Electron microscopy immunoprecipitation showed that TDP-43-containing nuclear complexes formed short fibers and oligomers ranging from 6 to 43 nm in diameter (FIG. 6c). Arrowheads indicated representative high-magnification images of TDP-43 complexes in FIGS. 6d-6f and 6h-6j. TDP-43 polymers displayed a short, tubular, non-parallel organization (FIGS. 6d, 6e and 6f). The length of major TDP-43-containing polymers was 150-350 nm. The representative image provided in FIG. 6f shows two branches of an isolated polymer that may be undergoing polymerization. The TDP-43-containing polymers were similar to in vitro baicalein-inducing TDP-43 polymers that formed a loosened F-actin-like assembly, but their morphology included irregular shapes and heterogeneity. Of note, linear immunogold labeling of TDP-43 was also observed in the nucleus (FIG. 6g). Remarkably, we observed a fibrogranular network composed of branched, knobbed filaments that was associated with globular structures, similar to known fibrogranular ribonucleoprotein, which was precipitated with TDP-43 antibodies (FIGS. 6k and 6l). No similar structure was precipitated using TIAR or CBP antibodies (data not shown). To carefully validate whether TDP-43 proteins constitute the fibrogranular network in the nucleus, we performed immunofluorescence experiments with a long antibody incubation period (4° C., 16-18 h) and used fluorescence microscopy with a resolution of 120 nm. The immunofluorescence analyses consistently revealed a filamentous network of TDP-43, in which TDP-43 was distributed along extended filaments and dense aggregates, as shown in FIG. 6k (FIG. 6m). Additionally, GFP-mTDP-43-FL proteins, similar to endogenous TDP-43, appeared in branched knobbed filaments associated with globular structures but that mTDP-43-PLDΔ proteins did not form globular structures and have reduced filaments (FIG. 6n). This result indicated that prion-like domain of TDP-43 is required to sequester TDP-43 into knobbed filaments. Rare GFP-mTDP-43-PLDΔ proteins appeared in filaments, potentially due to sequestration by endogenous TDP-43 via RNA binding domains. Herein, we identified the basic building blocks of cellular TDP-43 proteins, including oligomeric complexes, loosened filamentous polymers, and fibrogranular networks.

### Example 7: Rescue of Premature Aging by Correcting Aberrant Phase of Prion-Like Proteins

To further characterize whether TDP-43 proteins are present in the nuclear matrix, consisting of proteins such as lamina A/C, we double-stained TDP-43 (green) with lamina A/C (red) (FIG. 7). The TDP-43 fibrogranular framework was partially connected to lamina A/C (FIG. 7a, the arrow indicates the colocalization area). As defective lamin A can cause progeria and the a premature aging disorder HGPS, overexpression of progeria proteins is considered a cell model of aging and allowed us to explore the mechanistic link between TDP-43 and aging or a lamin A pathology. We thus overexpressed progeria proteins and examined TDP-43 localization, the efficiency of TDP-43PLD-mediated exon 9 skipping and TDP-43 polymers. In progeria-expressing cells, we found that TDP-43 proteins exhibited a pattern of diffusion and cytoplasmic mislocalization and failed to promote CFTR exon 9 skipping (FIGS. 7b and 7c, respectively). Significantly, in a few cells, we observed cytosolic aggregates of TDP-43, which is a pathological hallmark of TDP-43 proteinopathies (FIG. 7b, arrowhead). Western blotting further suggested the failure of TDP-43 polymer assembly in progeria-expressing cells (FIG. 7d). We further tested whether a pharmacology chaperone of TDP-43 PLD, baicalein, could rescue progeria-induced dysfunction of TDP-43. As shown in FIG. 4E, baicalein significantly induced the retention of nuclear TDP-43 in cells expressing progeria proteins, and a statistical analysis is shown in FIG. 7f. An in vivo splicing assay revealed that baicalein rescued TDP-43 dysfunction caused by progeria in a dose-dependent manner (FIG. 7g). The restoring ratio of exon skipping by baicalein is from 1.31 to 1.91 with a dosage of baicalein from 10 μm to 50 μm (the ratio is indicated at the bottom of FIG. 7g).

These results indicated that the progeria mutation disturbs TDP-43 PLD-mediated alternative splicing possibly due to a failure in TDP-43 polymer assembly and TDP-43 dysfunction, leading to extensive changes in alternative splicing in patients with HGPS. These TDP-43 dysfunctions can be functionally corrected by baicalein. Interestingly, we found that baicalein not only restored the activity of TDP-43 but also corrected nuclear shape defects in HGPS (FIG. 7j; arrowhead shows rescued nuclei). This result is consistent with a previous report that nuclear envelope was disrupted by misfolded TDP-43 proteins.

We further analyzed the reciprocal relation of lamin A and TDP-43. We observed a dramatic alteration of TDP-43 species in cells expressing lamin A (FIG. 7i). The 54 kD- and 70 kD-TDP-43 species disappeared, but the assembly of insoluble 90 kD-TDP-43 proteins (arrowhead) was observed in cells overexpressing lamin A (FIG. 7i). However, we failed to identify a physical interaction between TDP-43 and lamin A/C by coimmunoprecipitation, although immunofluorescence revealed that TDP-43 and lamin A/C partially colocalized in the punta (FIG. 7a). Perhaps TDP-43 interacts with lamin A via lamin B (SEQ ID No. 10), as lamin B acts as a prion-like protein to bind to b-isox. Together, the pharmacological effects of baicalein in the rescue of laminopathy suggest a crucial role for prion-like folding of TDP-43 proteins in disease aging.

### Example 8: Intrinsic Prion-Like Propensity of SMN

SMN has been shown to concentrate in subnuclear bodies called Gems and are incorporated into cytosolic stress granules (SG) through interaction with a prion-like protein, TIA1. Lorson et al. further identified a modular self-oligomerization region in exon 6 of SMNJ, and the disease severity is inversely proportional to the intracellular concentration of oligomerization-competent SMN proteins and loss of Gems. These well-characterized molecular behaviors and properties of SMN can be illustrated by an intrinsic prion-like propensity that was recently discovered by our group and others. Thus, we hypothesized that the SMN protein harbors a prion-like domain in exon 6. Disruption of the prion-like folding of SMN leads to the loss of prion-like self-polymerization and results in the subsequent development of SMA pathology.

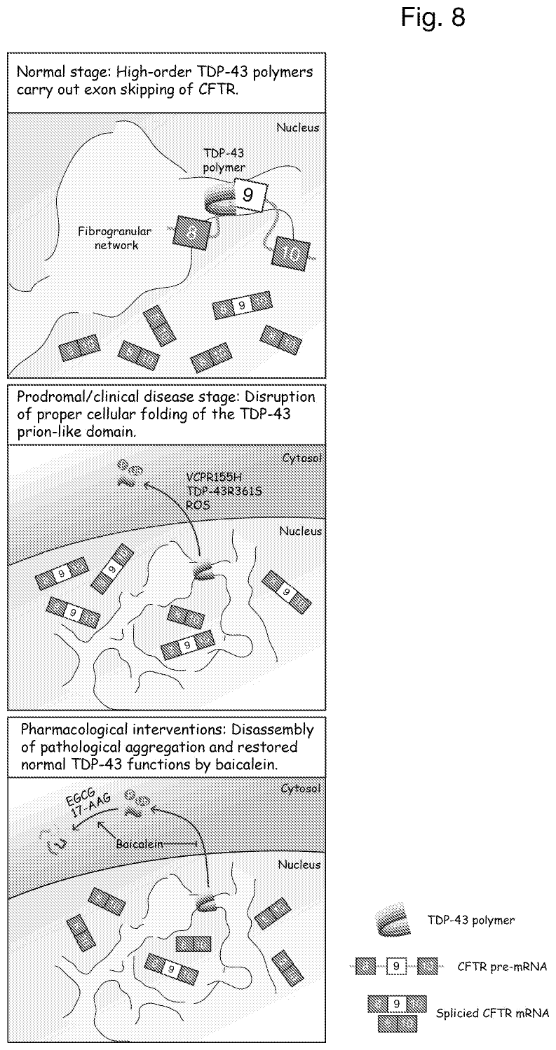

Biotinylated isoxazole (b-isox), a recently identified specific chemical probe, specifically recognizes the cross-β prion-like polymer and sequentially precipitates with proteins with low complexity, prion-like domains, or phase-separated domains, such as TDP-43 and Fus. We initially incubated 100 μM b-isox with cell lysates of mes23.5 and 293T cells at 4° C. to chemically precipitate whole prion-like proteins and then analyzed the efficiency of SMN binding by western blotting to assess the prion-like or phase transition potential of SMN (FIGS. 8a and b). Western blotting revealed the precipitation of both SMN and sumoylated SMN by b-isox (FIGS. 8a and b). Subcellular fractionation analysis further revealed that endogenous sumoylated SMN proteins were the predominant SMN conformations detected in insoluble urea fractions, and these proteins disappeared following b-isox treatments (FIG. 8c). H3K4me3 proteins were used as control for insoluble loading (FIG. 8c). Indeed, insoluble SMN has been reported to be important for the assembly of Cajal bodies (CBs) via the SUMO-like interacting motif (SIM-like). Thus, sumoylation is one of the cellular regulatory mechanisms that trigger nuclear SMN to form the separated phase though cross-β polymerization. Dysfunctional targeting of SMN to Gems and CBs has been suggested to be a feature of SMA in patients.

Unexpectedly, we found that the major conformation of SMN recognized by b-isox was located in the cytosol (FIG. 8b). Because visible SMN granules were not observed in the cytosol under normal conditions, we deduced that SMN polymers may participate in heteromeric interactions with other prion-like proteins to fulfill biological functions in the cytosol. Thus, we tested the phase transition potential of known SMN interactors by precipitating proteins with b-isox and found that PFN1, a known SMN interactor in the cytosol, precipitated with b-isox (FIG. 8b). Mutations in PFN1 cause co-aggregation with TDP-43 in patients with familial amyotrophic lateral sclerosis (ALS), supporting the hypothesis that PFN1 is a prion-like protein. In previous studies, PFN1 has been suggested to regulate cytoskeletal dynamics through direct interactions with the polyproline stretch in SMN, which is located in the self-oligomerization region. Thus, we postulated that these interactions were mediated by prion-like nature. Furthermore, we incubated full-length SMN or a panel of exon deletion constructs of SMN with b-isox and then performed a western blot analysis to identify the b-isox binding site in SMN. The map of SMN variants and results are shown in FIG. 8d. The regions encoded by exons 1-3, 4-7 and 6-7 precipitated well with b-isox, but the proline-rich domain and Gemin2 failed to precipitate with b-isox (FIG. 8d). The domains encoded by exons 2b and 6 have consistently been reported to self-interact. Although the domains encoded by both exon 2 and exon 6 display the potential to undergo phase transitions through cross-β polymerization, only fragments containing the region encoded by exon 6 form visible granules (FIG. 8f, arrowhead). Based on the results of the solubility examination, variants containing exons 6-7 were not detected in the insoluble fraction, although those variants formed visible granules. Thus, insolubility may not be proportional to granule formation. Both the N- and C-termini of SMN adopt a cross-0 polymer conformation, but the two domains of SMN have distinct biochemical properties and behaviors.

### Example 9: Increase of Cross-β Structures and Misfolded Protein Aggregates of SMN by SMA Disease-Causing Mutations and Exon 7 Deletion

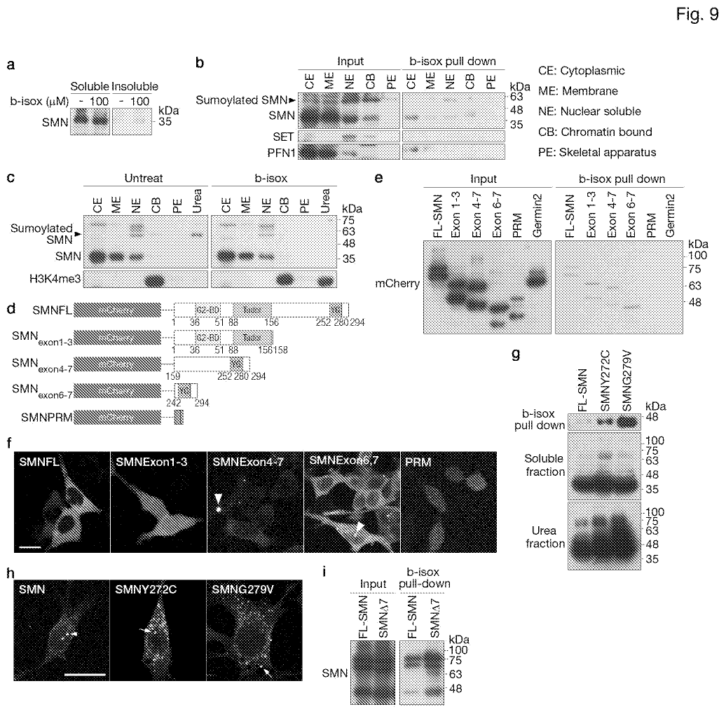

We tested whether the defect of prion-like folding of SMN correlated with SMA severity. We directly examined the cellular conformation of the patient-derived missense SMN mutants Y272C and G279V using b-isox. SMA type I mutations Y272C and G279V are point mutations in the YG box that have been shown to disrupt self-oligomerization. SMA type I is the most severe and common type, accounting for an estimated 50% to 70% of patients diagnosed with SMA. Compared to wild-type SMN, b-isox strongly precipitated the Y272C and G279V SMN proteins (FIG. 9a). Changes in the binding affinity of b-isox reflect that the cellular conformation of these two mutants had been altered. Interestingly, significant amounts of Y272C and G279V SMN proteins accumulated in the insoluble urea fraction, similar to pathological misfolded aggregates of the TDP-43 C-terminus (FIG. 9a). We further analyzed the localization of the two disease-causing mutants to determine whether these two mutants form pathological-like inclusions. Indeed, increased visible protein aggregates were observed in cultured motor neurons expressing Y272C and G279V SMN proteins (FIG. 9b). Thus, Y272C and G279V mutations altered the original functional conformations and lead to SMN misfolding. Misfolded protein aggregates of mutant Y272C or G279V SMN proteins have not been reported in patients with SMA. We deduced that overexpression of Y272C and G279V SMN proteins resulted in misfolded proteins that overloaded the capacity of the protein degradation system. This inefficient protein clearance leads to protein aggregation and allows us to detect distinct properties and behaviors of the mutants at the molecular level.

Furthermore, overexpression of the SMNA7 protein results in the formation of cytoplasmic and nuclear aggregates, which prompted us to test whether the conformation of SMNA7 differs from full-length SMN. Indeed, the precipitation of SMNA7 proteins with b-isox was increased compared to SMN, similar to the two SMA mutants described above (FIG. 9c). Based on these results, amino acids 272 and 279 and exon 7 are critical for the protein to adopt a competent prion-like conformation. The failure of SMN to adopt competent prion-like fold thus represents a misfolded protein, which are usually subjected to degradation by cellular clearance via lysosomes and autophagosomes, ultimately leading to the development of conformational disease. This phenomenon explains why SMNA7 is an unstable protein. The rapid degradation of SMNA7 proteins has been reported and is considered a major limiting factor that compensates for SMN function and results in cell death in patients with SMA.

### Example 10: Functional Rescue of SMNA7-Expressing Neurons and SMA Mice by Baicalein

We investigate whether baicalein can act as a pharmacological chaperone to refold misfolded SMNA7 into a functional prion-like domain, as shown in FIG. 8 (FIG. 10a). Consistently, baicalein decreased the formation of SMNA7 aggregates in 293T cells in a dose-dependent manner and increased the viability of SMNA7 cells by approximately 2-fold (FIGS. 10b and 10c). Interestingly, baicalein increased the neurite-like structure of SMNA7 cells (FIG. 10d). Conversely, SMNA7 cells became round and detached after treating with b-isox. Furthermore, co-immunoprecipitation analyses revealed that in the presence of baicalein, the interactions between PFN1 and SMNA7 were significantly increased (FIG. 10e). PFN1 is a known SMN interactor in the cytosol. We further examined whether baicalein attenuated the degradation of the SMNA7 protein. Indeed, baicalein significantly reduced SMNA7 degradation (FIG. 10f, arrow). Additionally, in a previous study, neurons transfected with SMNA7 extended significantly shorter neurites. We found baicalein increased the length of axons from NSC34 cells expressing SMNA7 by approximately 2-fold (FIG. 10g). The statistical analysis is shown in FIG. 10h. Based on these results, baicalein, which restores the prion-like bioactivity of misfolded TDP-43, also restores the prion-like functional deficiency in SMNA7.

A mouse model of SMA received daily intraperitoneal injections of baicalein (13.6 mg/kg/d) from birth, and we then assessed the animals using motor function tests and survival analyses to determine whether the in vitro findings were recapitulated in a mouse model in vivo (FIG. 10i). However, after baicalein treatment, the functional performance of SMA mice, including righting time, tube score, and tilting score, was improved at the 6th postnatal day (p<0.05), and the results were similar to heterozygous littermates treated with or without baicalein. At the 8^{th} postnatal day, the functional performance of the baicalein-treated SMA mice was better than that of the control SMA mice in the tube test (p=0.009) but not in the turnover test (p=0.065) or negative geotaxis test (p=0.58) (FIG. 10i). Our studies provide a method for restoring the functional SMN protein by modulating the folding of the SMNA7 C-terminus and SMA-associated mutants to ensure that the protein performs the cellular functions of full-length SMN.

### Example 11: Rescuing SMNA7-Expressing Neurons by Overexpressing Prion-Like Domain of TDP-43

We increased the amount of functional prion-like domains by overexpressing the TDP-43 prion-like domain (TDP-43-PLD) in NSC34 motor neuron cells expressing the SMNA7 proteins to confirm that the reduced level of the functional prion-like conformer caused axon degeneration in SMNΔ7-expressing neurons. The TDP-43-PLD is expected to adopt a common, structurally similar β sheet to compensate for the prion-like function of SMN through hetero-polymerization. Our experiment showed an increase in the axon length in cells expressing both SMNΔ7 and GFP-TDP-43 PLD compared to the GFP- and GFP-NPLD-expressing controls (FIG. 11a, arrowheads). The statistical analysis is shown in FIG. 11b. Consistently, SMN overexpression in motor neurons has also been shown to slow the onset of ALS and pathological symptoms in a model of mutant TDP-43. Thus, the level of the prion-like conformer is critical for motor neuron survival, and other functionally unrelated prion-like proteins can compensate for the function of the defective protein.

### Example 12: The Prion-Like Conformer-Based Therapeutic Strategy

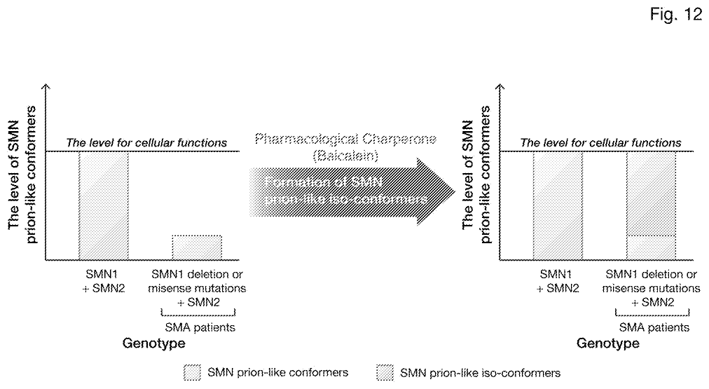

Based on the unique role of prion-like conformers in motor neurons, we proposed a therapeutic model of SMA, “the prion-like conformer-based therapeutic strategy”, in which partially misfolded SMA disease-causing mutants and SMNΔ7 are converted into prion-like folded proteins (FIG. 12). Baicalein enabled SMN mutants and SMNΔ7 to regain prion-like activity, subsequently increasing SMN-PFN1 interactions, reducing protein degradation, promoting the neurite-like outgrowth and survival of motor neurons, and improving motor function in SMA mice. The reassembled prion-like conformers of SMN mutants and SMNΔ7 by pharmacological chaperone were termed prion-like iso-conformers.

### Example 13: Simultaneously Heterologous Cross-β Templating of LC Sequence Domains Inside Cells

To test whether the soluble cross-β conformer of prion-like proteins is capable of self-replication, we systematically examined the levels of cellular cross-0 conformers of prion-like proteins after inducing the overexpression of a particular prion-like protein, followed by biotinylated isoxazole (b-isox) precipitation (FIG. 13). B-isox, a recently identified specific chemical probe, specifically coprecipitated with the cross-β prion-like polymer of the LC domain. We incubated 100 μM b-isox with a cell lysate of Htt-97Q-overexpressing 293T cells at 4° C. to chemically precipitate cross-β polymers and then analyzed the binding efficiency of prion-like proteins by western blotting (FIG. 13a-c). Htt-97Q in transfected cells before harvesting is shown in FIG. 1A. Although Htt-97Q protein forms visible aggregates, most Htt-97Q protein molecules are soluble (FIG. 13b). Significantly, in comparison to the control, we found that b-isox increased the precipitated amounts of TDP-43 and PFN1 prion-like proteins but did not change the precipitation efficiency of SMN and lamin B1 prion-like proteins in Htt97Q-overexpressing cells (FIG. 13c). Furthermore, based on the ability to template cross-β conformation, we defined this type of LC domain as a cross-β-nucleating domain.

As several LC proteins, including TDP-43 and Fus, contain RNA-binding domains, we tested the effect of RNA on the cross-β folding and templating of TDP-43. TDP-43 residues 147 and 149 have been shown to be critical for nucleic acid binding. Our previous work has further shown that the RNA-binding-deficient mutant TDP-43-F147/149L aggregated into soluble visible granules in the nucleus, which indicated that the loss of RNA binding induces phase separation through the structural conversion of the prion-like domain of TDP-43. To in vivo examine the cellular structure and templating ability of the RNA-binding-deficient mutant TDP-43-F147/149L, we incubated RNA-binding-deficient mutant proteins of TDP-43 with b-isox, followed by western blotting (FIG. 13d). Unexpectedly, similar to the prion-like domain deletion mutant of TDP-43, mTDP-43-PLDA, the RNA-binding-deficient mutant lost the capability of forming a cross-β conformer and template folding of PFN1 and Lam B, although it formed visible granules (FIG. 13d). These results implied that RNAs promoted the adoption of the cross-β conformation in TDP-43 and sequentially recruited a subgroup of prion-like proteins for conversion into the cross-0 conformers underlying physiological condition (FIG. 13d). Additionally, we deduced unlike stress granules, the visible granules of RNA-unbound TDP-43 assemble into a separate phase via a distinct conformation and cross-β-independent mechanism.

Next, we constructed the SUMOylation-defective TDP-43 mutant K136R and examined its in vivo capability of cross-β templating. Our experiments showed that in comparison with hTDP-43, the hTDPK136R mutant strongly bound to b-isox and significantly increased the cross-β conformer of endogenous TDP-43 and the prion-like proteins Lam B and PFN1 (FIG. 13e). Consistent to the results in FIGS. 13c and d, we found the capability of b-isox binding of LC domain is proportional to the templating ability of cross-β. Using anti-Flag or Lam B antibodies, immunoprecipitation analysis further revealed that hTDPK136R increased the association with Lam B (FIG. 13f). Accordingly, we found that hTDPK136R preferred to localize to the nuclear membrane where Lam B localizes. In comparison to ˜24% nuclear membrane localization of hTDP-43 FL, ˜70% hTDPK136R localized at the nuclear membrane (FIG. 13g). The dynamics of post-translational modification, such as SUMOylation, may be a structural regulator of nuclear architecture to direct gene expression through the conversion of prion-like protein folding.

To investigate the de novo mechanism of heterotypic interactions of the cross-β, we incubated TDP-43 and Lam B recombinant proteins with or without b-isox for 2 hr in vitro. We found that TDP-43 and Lam B in vitro assembled into short cylindrical filaments, which were disrupted in the presence of b-isox (FIG. 13h). This finding suggested that the cross-β interactions of TDP-43 and Lam B. Accordingly, we incubated TDP-43 and Lam B recombinant proteins separately for 1 hr and then mixed two recombinant proteins following by 1 hr incubation. We found that TDP-43 and Lam B formed oligomer-like structure and failed to assembly of short cylindrical filaments (FIG. 13h). This finding suggested that monomeric LC domain is a prerequisite for heterotypic cross-β interactions of prion-like proteins and may explain the formation of cross-β conformers was not proportional to the level of prion-like proteins in subcellular compartments (FIG. 13i). In cells, the percentage of cross-β conformers of LC protein was approximately 10˜70% by the b-isox binding analysis, e.g. 21.2% cross-β conformers of TDP-43 in Mes23.5 dopaminergic neurons (FIG. 13j). We assume that inside the cells, most of the intrinsically disordered LC domains are protected through structure folding or binding with other proteins that limits the cross-β-nucleating reactions. We suggested heterologous propagation of soluble cross-β occurs in cells, while monomeric LC domain was released or newly synthesized.

### Example 14: Loss of Cross-β Synchronization and Interactions of Disease-Causing Proteins Under a Pathological State of ALS

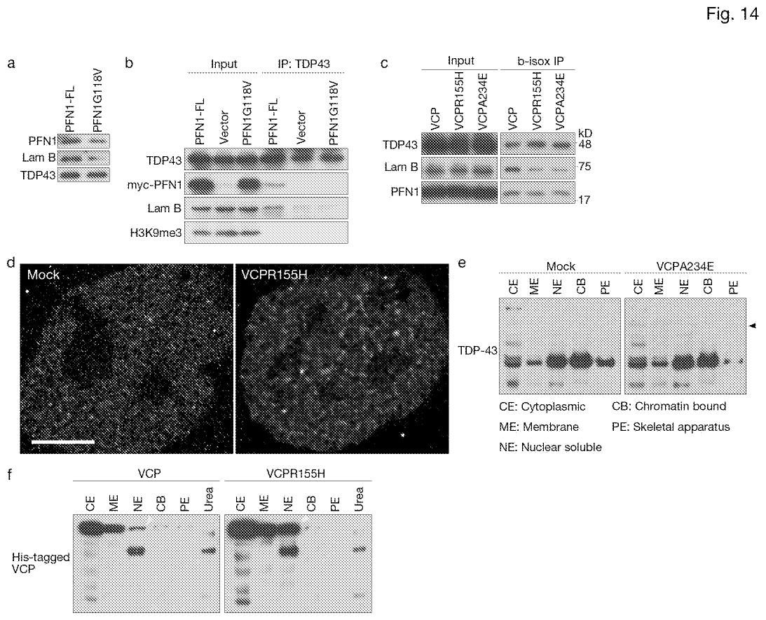

Next, we incubated the lysates of 293T cells overexpressing either wild-type PFN1 or patient-derived mutant PFN1G118V with b-isox, followed by western blot analysis. In comparison to PFN1G118V, PFN1-FL strongly bound b-isox, which was correlated with an increase in the precipitation of endogenous PFN1 and Lam B; however, no change in the precipitation of TDP-43 protein was observed (FIG. 14a). These results suggested that an increase in the cross-β conformer of prion-like proteins increased a selective subset of soluble cross-β conformers of hetero- or homotypic prion-like proteins. Notably, the G118V disease mutation impaired the ability of PFN1 to fold and propagate the cross-0 structure.

Given the self-interactions of prion-like domains, we further validated the heterointeracting partners of PFN1-FL and PFN1-G118V by immunoprecipitation. Our experiments showed that the association of TDP-43 with PFN1 or Lam B was increased when PFN1-FL was overexpressed but was not changed in the control or the PFN1-G118V mutant (FIG. 14b). A positive correlation of the amounts of soluble cross-β conformers of PFN1 and Lam B and their prion-like interactions with TDP-43 was observed. Altogether, the cross-β conformer of the LC domain can replicate in cells to initiate the de novo association network between LC proteins and this mechanism did not occur in a pathological state of PFN1-associated ALS. This result implied a potential relationship of cross-β polymerization defects to the etiology of ALS.

Additionally, using the b-isox precipitation assay to detect the in vivo cross-β conformation of TDP-43, we found that the cross-β binding affinity of b-isox with TDP-43 increased in cells overexpressing the ALS-associated VCP mutant VCP R155H, which is considered an in vitro disease model of ALS. (FIG. 14c). However, the cross-0 binding affinity of b-isox decreased with lamin b and PFN1 that suggested increased cross-0 of TDP-43 failed to propagation. We noticed that the nuclear architecture of TDP-43 was perturbed and observed the mislocalization of nuclear TDP-43 appeared in the cytosol of ALS-associated mutants (FIGS. 14d and e). We deduced that VCP R155H converted a portion of endogenous TDP-43 into disease-causing conformers with cross-β structure; therefore, reduces physiological cross-β of TDP-43. Reducing the physiological cross-β of TDP-43 leads to a decrease in cross-β interactions of other prion-like LC proteins, such as lamin b and PFN1, following by the breakdown of the TDP-43-PLD-structured residual framework in ALS-associated mutants.

The fractionation analysis further revealed that VCP R155H proteins were more stable than VCP wild-type proteins and that VCP R155H proteins were specifically increased in the nuclear chromatin-unbound fraction, namely, the nucleoplasmic fraction (FIG. 14f, arrowhead indicates VCP proteins in the nuclear chromatin-unbound fraction). Given that VCP has been suggested to act as a segregase, we assumed that underlying pathogenesis, ALS-associated VCP mutations increased the stability of nucleoplasmic VCP proteins, which may lead to the dissociation of TDP-43 from the nuclear structured framework, and sequentially mislocalization of nuclear TDP-43 proteins. Although the manner in which increased VCP activity converts the conformation of nuclear TDP-43 is not entirely clear, an important insight was provided by our results, suggesting a novel key etiology of ALS at the molecular level through deregulated physiological cross-β network.

### Example 15: A Proposed Model of Cellular Cross-β Self-Perpetuating

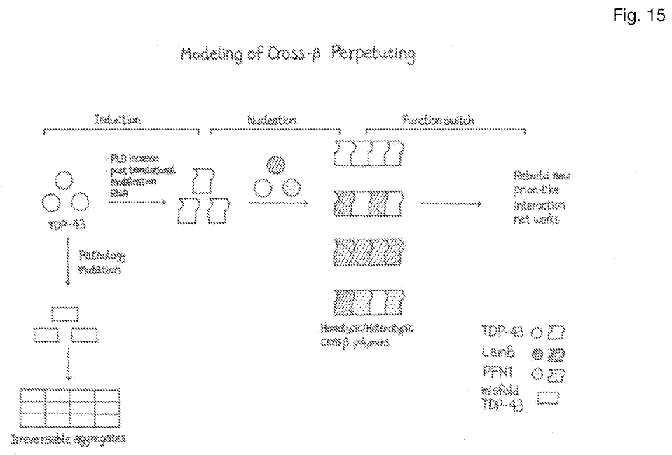

Currently known regulatory mechanisms of biological processes include the control of gene and protein expression, protein modification and noncoding RNAs by modulations in frequency, rate or extent. Herein, we uncovered a layer of regulation, in which a novel type of a β-sheet-rich domain is capable of structural replication by catalyzing self- or other protein conversion and sequentially forming biopolymers. Through the new prion-like network rebuilding or spatial reorganization, these naturally self-templated assembled biopolymers go beyond the functional and structural complexity to reset cellular homeostasis and cellular adaptations. We termed this biological reaction “cross-β self-perpetuating”. A proposed model of cross-β self-perpetuating is illustrated in FIG. 15. We divided this biological process into three stages: induction, synchronization and function switch. At the induction stage, an infectious conformation may be induced at the protein level by prion-like proteins, RNA or postmodification. These β-sheet-rich conformers bind to homo- or heterologous prion-like molecules and catalyze their conversion at the synchronization stage and, following polymeric assembly, rebuild a new prion-like interaction network at the function switch stage. In ALS patients with inherited proteinopathies, pathological mutations lose the ability to template cross-β polymers with a loss of normal cellular functions and a gain of irreversible pathological β-sheet-rich aggregates. This novel type of regulation can dramatically reshape cellular biochemistry by organizing the existing set of proteins without altering DNA or RNAs.

### Example 16: Horizontal Transmission of p53 Amyloidogenic Strains

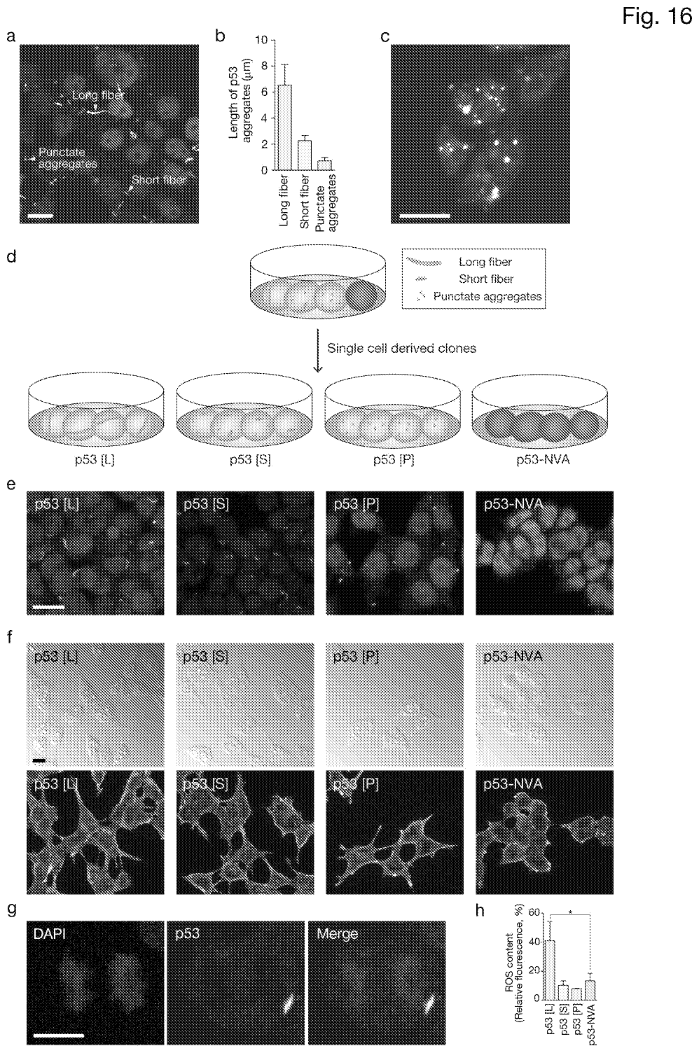

We observed a striking degree of heterogeneity among p53 aggregates in the cytosol of 293T cells by immunofluorescence with a p53 antibody, including long fibers (5-10 μm), short fibers (1.6-3.2 μm) and punctate aggregates (0.5-1 μm in diameter) (FIG. 16a, arrows). The length of the p53 fibers and p53 aggregates were analyzed (FIG. 16b). Consistent to the accumulation of amyloid deposits by disturbing protein degradation pathway, the MG132 protease inhibitor significantly increased p53 aggregates and converted the three types of p53 fibers into irregular inclusions (FIG. 16c). To determine whether these heterogeneous p53 amyloid patterns are inherited or are random conversions, we subcultured and isolated single colonies from heterogeneous pools of cells. The experimental flowchart is shown in FIG. 16d. After single clonal expansion, only one p53 amyloid pattern was observed in a single cell derived clone, which then stably expressed that specific phenotype. We obtained four phenotypes shown in FIG. 16a, including long and short fibers, puncta, and diffuse nuclear staining, and these phenotypes were stably propagated (FIG. 16e). We termed these p53 phenotypes of the isolated strains as follows: p53 [L] (Long fibers), p53 [S] (Short fibers), p53 [P] (Puncta) and p53-NVA (No Visible Aggregates). Cells with p53 aggregates showed better attachment and mesenchymal-like morphologies (FIG. 16f; corresponding actin staining below).

### Example 17: Inherited Oncogenicity and Cancer Stemness of p53 Amyloidogenic Strains

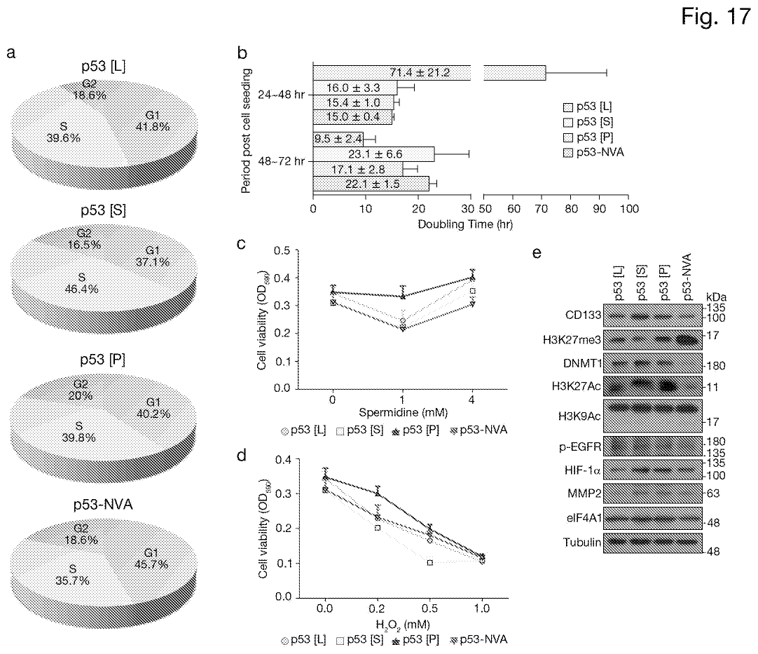

Our clonally expanded system provided an excellent cellular model to precisely investigate the oncogenicity of strain-specific p53 amyloid conformations in vivo. We analyzed cell growth of the four p53 amyloid strains by examining cell cycle flow and counting doubling time (FIGS. 17a and 17b). Flow cytometry analysis revealed increases in the percentage of S (synthesis) phase cells of 4.1%, 10.7% and 3.9% in the p53 [L], [S] and [P] cells, respectively (FIG. 17a). Consistent with the accelerated cell growth, we found that cells with cytosolic p53 aggregates, including p53 [L] and [P], have faster doubling times at 48-72 hr post-seeding (FIG. 17b). p53 [L] showed a delayed lag phase for 24 hr followed by a sudden sharp increase in slope (FIG. 17b). In contrast to the toxic amyloids observed in neurodegenerative contexts, p53 amyloids appear to promote cell growth. Additionally, to assess the stress response, the four cell strains were challenged with spermidine and H₂O₂ (FIGS. 17c and 17d). Cell viability was impaired under all stress conditions. However, the p53 [P] clone was somewhat resistant to the spermidine-induced effects on cell viability, and showed a higher tolerance toward oxidative stress (FIGS. 17c and 17d). These results suggest that these strain-specific p53 amyloid conformations have unique and long-lasting biochemical and physiological effects. To further identify key pathophysiology pathways involved in tumorigenesis of p53 aggregates' gain-of-function, we performed a serial screen by western blotting and compared the expression of cancer-related proteins in the four strains. Generally, p53 [S] and [P] showed similar expression profiles (FIG. 17e). Most significantly, the expression of a cancer stemness biomarker, CD133, was elevated in three of the p53 aggregate clones (FIG. 17e). Additionally, a group of epigenetic regulators altered their expression in present of p53 aggregates. H3K27me3, which is associated with epigenetic silencing, was dramatically reduced in p53 aggregate strains, but another silencing regulator, DNMT1 was increase in p53 aggregate strains (FIG. 17e). The epithelial-mesenchymal transition (EMT) related genes, including p-EGFR and HIF-1α also increased in in p53 aggregate strains that correlated with their morphology switch. These results reveal the first direct evidence of a role for p53 amyloids in the induction of cancer cell stemness, as well as for p53 amyloids in the downregulation of methylated histone H3, which could lead to global changes in gene expression. Together with the horizontal transmissibility shown in FIG. 16, the three endogenous p53 aggregates were able to switch into malignant cancer cells including activating EMT, increasing cancer stemness, and dramatically altering epigenetic regulators, and the strain-specific p53 amyloids can propagate oncogenic inheritance via a protein-based mechanism.

### Example 18: Infectivity of p53 Amyloid Strains

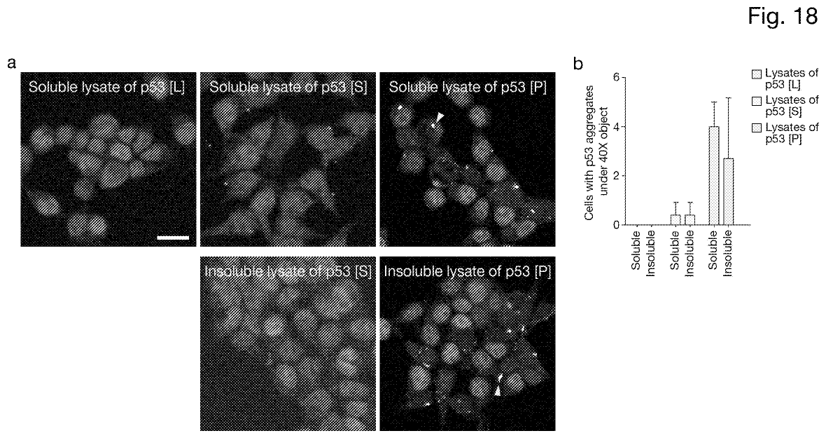

To assess whether naturally occurring p53 amyloids can infect cells and promote prion-like activities, cell extracts isolated from individual strain-specific p53 clones were added to p53-NVA recipient cells (FIG. 18). We produced extracts using a hypotonic buffer that caused cells to swell and separated the soluble and insoluble fractions by centrifugation. As determined using immunostaining with p53 antibody, extracts generated from the p53 [P] clone induced p53 aggregates in p53-NVA cells using either the soluble or the insoluble fractions; notably, all three types of p53 assemblies were found in the recipient cells, suggesting that the inheritability of p53 [P] was lost during the extraction process (FIG. 18a, arrowhead). No significant induction of p53 fibers was observed following the addition of extracts generated from either the p53 [L] or the p53 [S] strains, although few dot-fluorescence signal labeled on the cell periphery of p53 [S] (FIG. 18a). Failure of the endogenous p53 [L] and [S] extracts to induce fiber formation in p53-NVA cells was inconsistent with the results demonstrating the induction of in vitro p53 fibers. We speculated the aggregate size of p53 [L] and [S] could be too large to enter the recipient cells. A statistical analysis of induction efficiency is shown in FIG. 18b. Following the addition of p53 amyloid extracts to non-amyloid cells, we did not observe the prion inheritance patterns observed for three p53 strains in recipient cells, although p53 [P] can behave as an infectious entity.

### Example 19: An Interplay Between TDP-43 and p53 in p53 Amyloid-Positive Contents

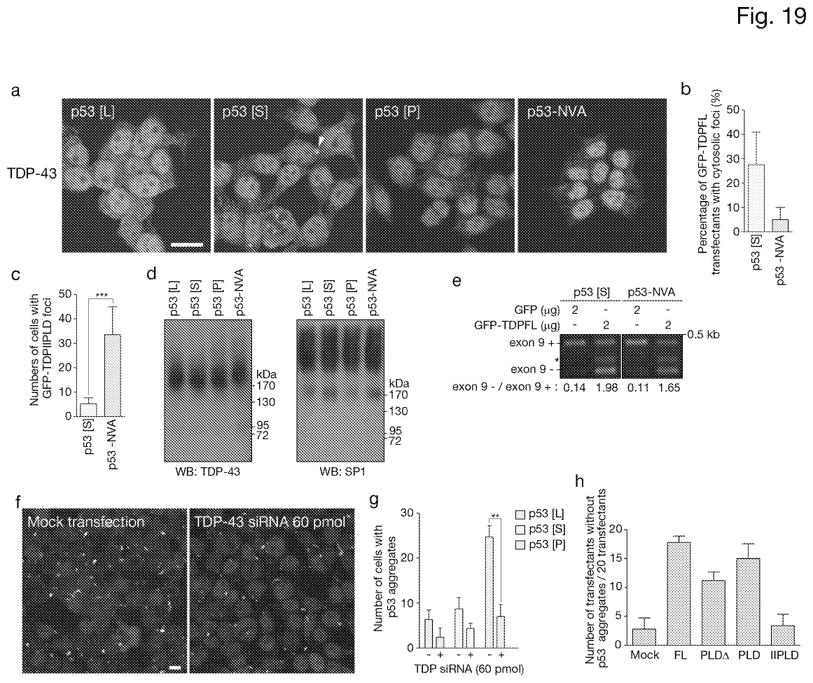

Prion or aggregation-prone proteins could induce secondary protein misfolding. Thus, we tested whether p53 aggregation affects TDP-43, a prion-like protein found in aggregates in the ubi-positive inclusions of patients with FTLD-U and ALS. We examined TDP-43 localization in four strains by fluorescence microscopy (FIG. 19a). We found TDP-43 proteins were sequestered into TDP-43 cytosolic foci specifically in p53 [S], suggesting p53 [S] amyloids modulated TDP-43 aggregation propensity (FIG. 19a; arrowhead). To carefully investigate the effects of p53 amyloid on functional and misfolded pathological-like TDP-43 proteins, we overexpressed the GFP-TDP-43-FL proteins or pathological-like GFP-TDP-4311P fragments in the p53 [S] or p53-NVA clones and then examined the localization of GFP-TDP-43 proteins (FIGS. 19b and 19c). The sequestration of TDP-43-FL into cytosolic foci was increased 6-fold in the p53 [S] clone compared with the p53-NVA clone, suggesting that particular folded amyloid could enhance functional TDP-43 aggregation (FIG. 19b). Conversely, formation of pathological-like inclusion of TDP-43 (GFP-TDP-4311P) was decreased 6-fold in the p53 [S] clone compared with the p53-NVA clone. We deduced perhaps p53 and TDP-43 may compete cellular factors involving in the formation of misfolded aggregation (FIG. 19c).

Furthermore, using native gel analysis, TDP-43 proteins extracted from the p53-NVA clone had higher molecular weights than those extracted from the p53 aggregate clones (FIG. 19d). A Q-rich protein, Sp1, was used as a control. To test whether p53 amyloids affect TDP-43-mediated biological process, we performed an in vivo alterative splicing assay on CFTR exon 9 skipping, which regulated by prion-like activities of TDP-43. More efficient CFTR exon 9 skipping was observed in the p53 [S] clone compared with the p53-NVA clone (FIG. 19e).

Additionally, a significant reduction in p53 amyloid fibers was found in cells transfected with TDP-43 siRNAs (FIG. 19f). Statistical analysis of p53 aggregate for the three p53 aggregate-phenotypes is shown in FIG. 19g. These results indicate a reciprocal interplay of aggregation propensity between p53 and TDP-43.

A significant reduction in p53 amyloid fibers was found in cells transfected with TDP-43 variants (FIG. 19h). Statistical analysis of p53 aggregate is shown in FIG. 19h. These results indicate manipulating the secondary aggregation-prone protein, such as TDP-43 in here, could modulate the aggregation propensity of p53.

### Example 20: Determination of p53 Aggregation Formation by HSPB1

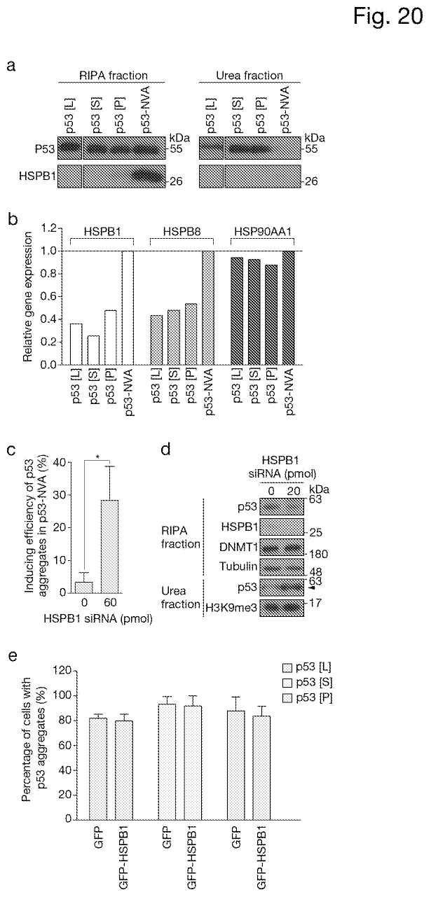

Western blotting and immunostaining analysis revealed the concomitant loss of HSPB1 expression in the p53 [L], [S], and [P] clones, but not in the p53-NVA clone (FIG. 20a). Consistent with the protein expression profiling of HSPB1 in the four strains, we found that HSPB1 mRNA expression decreased to 35%, 25.2% and 47.9% of normal levels in the p53 [L], [S], and [P] clones, respectively (FIG. 20b).

Interestingly, of the twenty-five heat shock proteins present on the microarray chip, only HSPB1 and HSPB8 showed decreased mRNA expression in p53 aggregate strains; the other heat shock proteins, including HSP70 and HSP90, did not display changes in mRNA expression (FIG. 20b; data not shown). Therefore, we tested whether decreased HSPB1 expression in the p53-NVA clone could induce p53 amyloid fibers using siRNA knockdown of HSPB1. Indeed, knockdown of HSPB1 induced p53 fibrils and punctate and the statistical analysis of p53-NVA cells with p53 aggregates is shown in FIG. 14c. Western blotting analysis further confirmed an increase in the amount of insoluble p53 proteins in the HSPB1 siRNA-knockdown cells (FIG. 20d).

Additionally, we found that H₂O₂ treatment, which also reduces HSPB1 expression, induced p53 aggregate formation. Of note, overexpression of HSPB1 in p53 [L], [S] or [P] clones did not significantly reduce p53 amyloids (FIG. 20e). Thus, HSPB1 is required for the maintenance of functional p53 but can't help to refold misfolded p53 proteins.

### Example 21: The Efficient Elimination of p53 Aggregates by Overexpression of p53 Proteins and an Aβ Amyloid Disassembling Agent

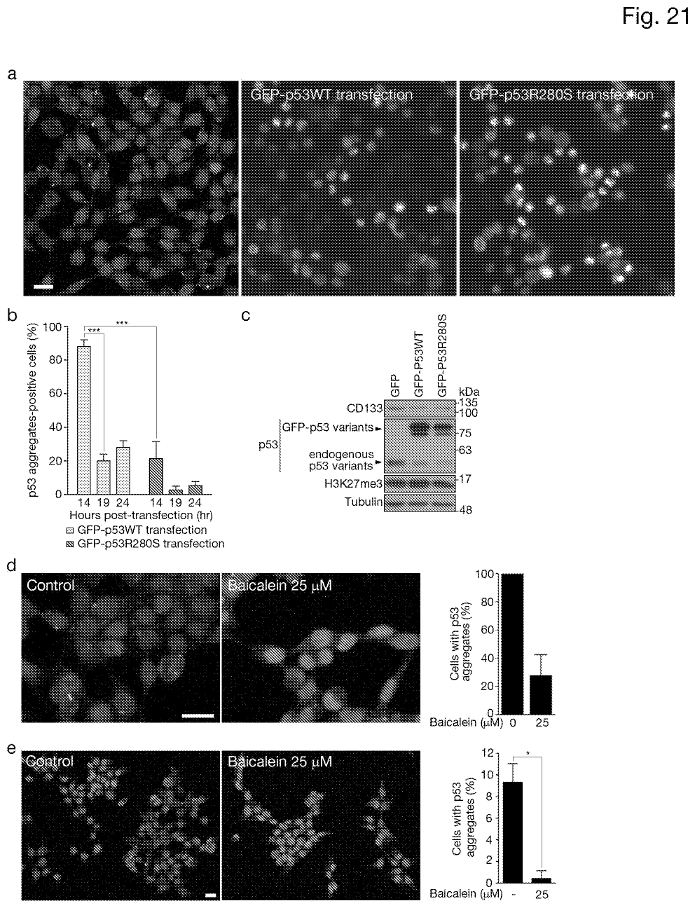

To monitor the behavior of p53 amyloids in living cells, we tested whether overexpression of p53 itself would be sequestered into p53 aggregates (FIG. 21). Unexpectedly, we found that exogenous GFP-p53WT proteins were not sequestered into strain-specific fibers nor did they show puncta staining (FIG. 21a). Wilde-type p53 overexpression efficiently removed p53 [L], [S] and [P] aggregates by up to 60%. We also found that a p53 mutant R280S (GFP-p53R280S) could almost completely eliminate p53 aggregates (FIG. 21b). The p53 amyloid clearance efficiencies were calculated and are summarized in FIG. 21b.

Remarkably, both overexpressed p53WT and R280S proteins not only reduced the expression of CD133, which was elevated while p53 aggregates and induced cell death (FIG. 21c).

Additionally, we treated 293T cells with an Aβ amyloid disassembling agent, baicalein, following immunofluorescence staining with anti-p53 antibodies. We found baicalein also reduced p53 misfolded aggregates and suppressed the spontaneous aggregation of p53 (FIGS. 21d and 21e). These results suggested that the Aβ amyloid disassembling compounds could potentially be used in cancer therapy.

### Example 22: Identification of Prion-Like LC Domain of Rb

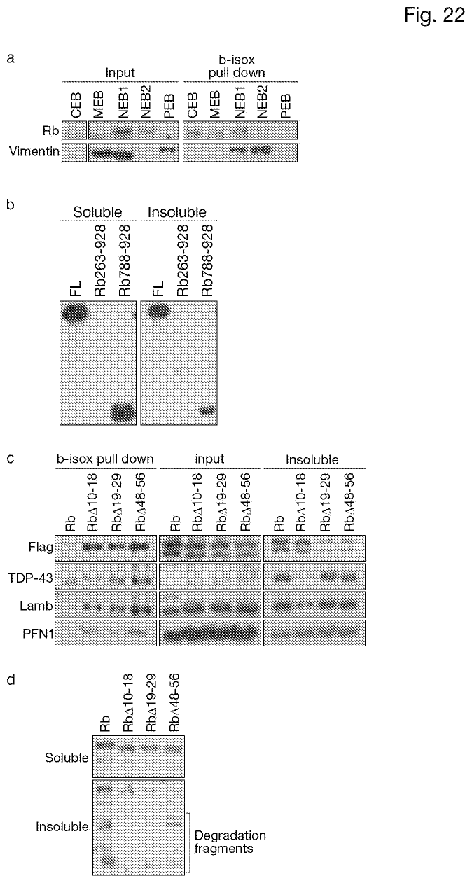

We incubated 100 μM b-isox with cell lysates of 293T cells at 4° C. to chemically precipitate whole prion-like proteins and then analyzed the efficiency of Rb binding by western blotting to assess the prion-like or phase transition potential of Rb (FIG. 22). Western blotting revealed the precipitation of Rb by b-isox (FIG. 22a). Solubility analysis further revealed that small pocket of Rb (aa.263-788) is prion-like domain (FIG. 22b). The N-terminus of Rb (aa. 10-56) stabilizes prion-like conformation (FIG. 22c). Deletion of Rb N-terminus leads to protein degradation (FIG. 22d).

1-21. (canceled)

22. A method for preventing or treating a conformational disease in a subject, comprising administering to the subject in need thereof an effective amount of a therapeutic agent selected from the group consisting of flavonoid, siRNA against HSP27, secondary aggregation-prone protein, a plasmid that expresses prion-like low complexity (LC) domain, a heat shock protein modulator and combination thereof, wherein conformation disease is degradative conformational disease, non-amyloid aggregation conformational diseases or amyloid aggregation conformational diseases selected from cancers with p53 aggregation, Down syndrome, or glaucoma.

23. The method of claim 22, wherein the flavonoid is baicalein or its derivative.

24. The method of claim 22, wherein the siRNA against HSP27 is at least 90 to 100% identical to SEQ ID NO:11, SEQ ID NO:12, SEQ ID NO: 13, SEQ ID NO: 14, SEQ ID NO: 15, SEQ ID NO: 16, SEQ ID NO:17 or SEQ ID NO:18.

25. The method of claim 22, wherein the secondary aggregation prone protein is TDP-43.

26. The method of claim 22, wherein the head shock protein modulator is 17-N-allylamino-17-demethoxygeldanamycin (17-AAG) or arimoclomol.

27. The method of claim 22, wherein the degradative conformational diseases is spinal muscular atrophy (SMA), childhood cancer, retinoblastoma, bladder cancer, breast cancer, osteogenic sarcoma and Rb (Rb1) deficient cancers.

28. The method of claim 22, wherein the non-amyloid aggregation conformational diseases is amyotrophic lateral sclerosis (ALS), frontotemporal lobar dementia with ubiquitin (FTDL-U), hippocampal sclerosis or mixed proteinopathy.

29. A method to increase the prion-like conformer of a prion-like low-complexity (LC) protein, by administering a flavnoid or a HSP27 siRNA.

30. The method of claim 29, wherein the flavonoid is baicalein or its derivative.

31. The method of claim 29, wherein the siRNA against HSP27 is at least 90 to 100% identical to SEQ ID NO:11, SEQ ID NO:12, SEQ ID NO: 13, SEQ ID NO: 14, SEQ ID NO: 15, SEQ ID NO: 16, SEQ ID NO:17 or SEQ ID NO:18.

32. A method to treat TDP-43 proteinopathy in a subject, comprising the step of administering a prion-like polymer.

33. The method of claim 32, wherein the TDP-43 proteinopathy is amyotrophic lateral sclerosis (ALS), frontotemporal lobar dementia with ubiquitin (FTDL-U), milder cognition impairments (MCI), Alzheimer's' disease (AD) and mixed pathology of neurodegeneration.
